(19) United States
(12) Patent Application Publication
(10) Pub. No.: US 2018/0001174 A1
(43) Pub. Date: Jan. 4, 2018

(54) WEARABLE DEVICE, CONTROL METHOD, AND PROGRAM

(71) Applicant: SEIKO EPSON CORPORATION, Tokyo (JP)

(72) Inventors: Ichiro AOSHIMA, Matsumoto-shi (JP); Yusuke TAKAHASHI, Matsumoto-shi (JP); Hironori HASEI, Azumino-shi (JP)

(73) Assignee: SEIKO EPSON CORPORATION, Tokyo (JP)

(21) Appl. No.: 15/629,213

(22) Filed: Jun. 21, 2017

(30) Foreign Application Priority Data
Jul. 1, 2016 (JP) 2016-131384

(51) Int. Cl. A63B 69/00; A61B 5/024; A61B 5/00; A63B 71/06

(52) U.S. Cl. CPC A63B 69/0028; A61B 5/6824; A61B 5/02444; A63B 2071/0663; A61B 2562/0219

(57) ABSTRACT

A wearable device includes: a biological information detection unit which detects biological information of a user; a body motion detection unit which detects a body motion signal related to a movement of the user; a processing unit which generates notification information based on at least one type of information, of the biological information and the body motion signal; and a notification unit (display unit) which notifies the user of the notification information. The processing unit detects a movement state based on the body motion signal of the user. The processing unit performs processing (control) in which the notification unit is made to notify the user of the notification information if a first movement state included in the movement state is detected. The processing unit performs processing (control) in which the notification of the notification information by the notification unit is stopped if a second movement state is detected.

### CROSS-REFERENCE TO RELATED APPLICATION

The current application claims priority to Japanese patent application 2016-131384, filed Jul. 1, 2016, the entirety of which is incorporated herein by reference.

### BACKGROUND

### 1. Technical Field

The present invention relates to a wearable device, a control method, and a program.

### 2. Related Art

A wearable device which is worn by a user and displays information of pulse rate, exercise intensity and the like measured during an exercise such as walking or running has traditionally been known. The wearable device disclosed in JP-A-2009-34366 constantly displays biological information such as pulse rate measured during an exercise, on a display device such as a liquid crystal device. If measurement cannot be performed, “- -” or the like is displayed indicating that measurement is impossible (measurement cannot be performed). In the wearable device, each component is driven by electricity supplied from a built-in battery. Therefore, unnecessary electricity consumption needs to be restrained as much as possible in order to perform measurement for a long time.

JP-A-2009-34366 discloses a configuration for restraining measurement processing and display processing in the case where it is difficult to measure pulse rate or the like. However, JP-A-2009-34366 includes no description about how to control the device when the user does not or cannot view the display even in the state where the measurement of pulse rate or the like is possible. That is, it is unnecessary to display information on the screen of the wearable device when the user does not or cannot view the screen, because the user will not visually recognize the information. Moreover, driving the display device to display the information causes unnecessary electricity consumption.

Also, a wearable device which provides convenience for the user to be able to browse information on the wearable device when intended and functions over a longer period of time by reducing its electricity consumption has been looked for.

### SUMMARY

An advantage of some aspects of the invention is to provide a wearable device which can properly communicate information measured during an exercise to the user, based on the exercise state of the user, and can also restrain electricity consumption.

### Application Example 1

A wearable device according to this application example includes: a biological information detection unit which detects biological information of a user; a body motion detection unit which detects a body motion signal related to a movement of the user; a processing unit which generates notification information based on at least one type of information, of the biological information and the body motion signal; and a notification unit which notifies the user of the notification information. The processing unit detects a movement state based on the body motion signal of the user. The processing unit performs processing in which the notification unit is made to notify the user of the notification information if a first movement state included in the movement state is detected. The processing unit performs processing in which the notification of the notification information by the notification unit is stopped if the movement state that is different from the first movement state is detected.

According to this application example, the processing unit stops the notification of the notification information by the notification unit if the movement state that is different from the first movement state is detected. Therefore, the consumption of driving electricity that is needed to notify the user of the notification information during this time can be restrained.

Also, if the processing unit detects the first movement state based on the body motion signal, the notification unit notifies the user of the notification information. Therefore, the user can recognize the notification information in the first movement state. For example, in the case where the notification unit is a display device, the state where the user can visually recognize the display device during an exercise is detected as the first movement state. The notification information displayed on the display device is highly likely to be visually recognized by the user and therefore can be easily communicated to the user. Meanwhile, if the movement state that is different from the first movement state, such as the state where the user cannot visually recognize the display device because the user is concentrating on the exercise, is detected, the display device is switched to a display-off state and therefore unnecessary electricity consumption is eliminated. Thus, based on the exercise state of the user, information measured during the exercise can be properly communicated to the user and electricity consumption can be restrained.

### Application Example 2

The processing unit described in the foregoing application example may stop the notification of the notification information if a first period has passed after the processing in which the notification unit is made to notify the user of the notification information is performed.

According to this application example, the electricity consumption for the notification processing after the lapse of the first period can be reduced.

### Application Example 3

The processing unit described in the foregoing application example may stop the notification of the notification information by the notification unit if a transition from the first movement state that is detected to a second movement state is detected.

According to this application example, the electricity consumption for the notification processing after the transition to the second movement state can be reduced.

### Application Example 4

The first movement state described in the foregoing application example may include a static movement in swimming.

According to this application example, the static movement in swimming is not a dynamic movement like a swimming movement during swimming. Therefore, in the static movement state, the user is more likely to notice the presence/absence of the notification information.

### Application Example 5

The second movement state described in the foregoing application example may include at least one of swimming movement, water walking, and water aerobics.

According to this application example, during an exercise with a swimming movement, water walking or water aerobics, the user is concentrating on the exercise. Therefore, it can be said that this is a movement state where the user cannot easily notice the notification information even if the notification information is provided.

### Application Example 6

The biological information detected by the biological information detection unit described in the foregoing application example may include at least pulse rate.

According to this application example, the state of biological load that changes according to the movement state of the user can be grasped by the detection of pulse rate.

### Application Example 7

The notification information described in the foregoing application example may includes at least one type of information, of at least one of maximum pulse rate and average pulse rate over a one-way distance from a previous turn movement to a current turn movement, a number of strokes taken, a stroke pitch, a stroke length, and a swimming time.

According to this application example, the user can be notified of information about the movement state over the one-way distance carried out by the user immediately before.

### Application Example 8

The notification information described in the foregoing application example may include at least one type of information, of a cumulative number of turns taken and a cumulative swimming distance.

According to this application example, the user can be notified of information about the cumulative movement state carried out by the user.

### Application Example 9

The body motion detection unit described in the foregoing application example may include at least one of an acceleration sensor, a pressure sensor, and a gyro sensor.

According to this application example, a body motion signal about the movement state of the user can be detected.

### Application Example 10

An entry into the water and an exit from the water of the wearable device worn by the user may be detected, using the pressure sensor described in the foregoing application example.

According to this application example, the pressure difference between the water pressure and the air pressure can be clearly distinguished from each other by the pressure sensor. Therefore, the entry into the water and the exit from the water can be detected.

### Application Example 11

The processing unit described in the foregoing application example may perform processing in which the notification unit is made to notify the user of summary information if a second period has passed after a transition of the wearable device from the entry into the water to the exit from the water.

According to this application example, after an exercise in the water is finished, the user can be notified of the summary information and thus can be made to recognize the summary information.

### Application Example 12

The summary information described in the foregoing application example may include at least one of an average pulse rate, a maximum pulse rate, a physical strength evaluation, a training effect, and degree-of-fatigue information, and at least one of a swimming time, a swimming distance, a number of turns taken, a swimming style, calories burned, an average SWOLF, and a best time recorded over a one-way distance between turn movements.

According to this application example, the result of comprehensive analysis about the movement state carried out by the user can be displayed.

### Application Example 13

A wearable device according to this application example includes: a biological information detection unit which detects biological information of a user; a body motion detection unit which detects a body motion signal related to a movement of the user; a processing unit which generates notification information based on at least one type of information, of the biological information and the body motion signal; and a notification unit which notifies the user of the notification information. The processing unit determines an exercise type carried out by the user, based on the body motion signal detected by the body motion detection unit. If an exercise of the exercise type is continued for a third period or longer and a first movement state is detected based on the body motion signal, the processing unit acquires the biological information detected by the biological information detection unit in the first movement state, generates relation information between the exercise type and the biological information that is acquired, and estimates the biological information based on the relation information.

According to this application example, the relation information between the exercise type determined based on the body motion signal and the biological information detected in the first movement state is generated. The biological information is estimated based on the relation information. Therefore, for example, even if the biological information cannot be detected in the third period in which the exercise of the exercise type is carried out, the biological information in the third period can be estimated referring to the generated relation information.

### Application Example 14

The processing unit described in the foregoing application example may generate information indicating a remaining time before reaching the third period.

According to this application example, for example, if the user is notified of the information indicating the remaining time, the user can learn the time period for which the exercise should be continued. Since the relation information is generated if the exercise of the exercise type is continued for the third period or longer, the relation information corresponding to the exercise type can be increased.

### Application Example 15

The notification unit described in the foregoing application example may notify the user by at least one of vibration or audio output when the third period is reached.

According to this application example, the user is more likely to notice that the third period is reached, via a stimulus of vibration to the body during the exercise or via an audio output to the auditory sense.

### Application Example 16

The processing unit described in the foregoing application example may generate the notification information including a time period for which the user is made to maintain the first movement state, and may cause the notification unit to notify the user of the notification information.

According to this application example, the opportunities where the user, notified of the time period for which the first movement state is to be maintained, maintains the first movement state increase. Therefore, more reliable biological information is more likely to be detected. The exercise type and the corresponding biological information stored in the relation information can be increased.

### Application Example 17

The wearable device described in the foregoing application example may further include a storage unit which stores the relation information between an exercise intensity of the exercise type and the biological information detected in the first movement state, in association with the exercise type.

According to this application example, the relation information related to each exercise type can be stored.

### Application Example 18

The processing unit described in the foregoing application example may decide the biological information of the user at the current time, based on the body motion signal of the user detected by the body motion detection unit, with reference to the relation information.

According to this application example, the current biological information of the user can be decided from the current body motion signal of the user, with reference to the stored relation information. For example, even if the biological information during the exercise of the user is not detected by the biological information detection unit, the biological information can be decided, simply based on the body motion signal detected by the body motion detection unit.

### Application Example 19

The exercise type described in the foregoing application example may include at least one type of front crawl, breaststroke, backstroke, butterfly stroke, and water walking.

According to this application example, the device can cope with various swimming styles.

### Application Example 20

A control method according to this application example includes a notification control method and a generation method. The notification control method includes: acquiring biological information about a user; acquiring a body motion signal relating to a movement of the user; performing notification processing in which the user is notified of notification information to notify the user of; generating the notification information, based on at least one type of information, of the biological information and the body motion signal; detecting a movement state based on the body motion signal of the user and performing processing in which the notification of the notification information is executed by the notification processing if a first movement state included in the movement state is detected; and performing processing in which the notification of the notification information is stopped if the movement state that is different from the first movement state is detected. The generation method includes: determining an exercise type carried out by the user, based on the body motion signal; acquiring the biological information in the first movement state, if an exercise of the exercise type is continued for a third period or longer and the first movement state is detected based on the body motion signal; and generating relation information between the biological information that is acquired and the exercise type that is determined.

According to this application example, the notification of the notification information is stopped if the movement state that is different from the first movement state is detected. Therefore, the consumption of driving electricity that is needed to notify the user of the notification information during this time can be restrained.

Also, if the first movement state is detected, the user is notified of the notification information. Therefore, the user can recognize the notification information in the first movement state. For example, if the first movement state is defined as the state where the user is not exercising, and the state that is different from the first movement state is defined as the state where the user is exercising, the user in the first movement state is not concentrating on the exercise and therefore is highly likely to notice the notification information and the information can easily communicated to the user. Meanwhile, if the movement state that is different from the first movement state is detected, electricity consumption is restrained. Therefore, based on the exercise state of the user, information measured during the exercise can be properly communicated to the user and electricity consumption can be restrained.

### Application Example 21

A program according to this application example causes a computer to execute a notification control method and a generation method. The notification control method includes: acquiring biological information about a user; acquiring a body motion signal relating to a movement of the user; performing notification processing in which the user is notified of notification information to notify the user of; generating the notification information, based on at least one type of information, of the biological information and the body motion signal; detecting a movement state based on the body motion signal of the user and performing processing in which the notification of the notification information is executed by the notification processing if a first movement state included in the movement state is detected; and performing processing in which the notification of the notification information is stopped if the movement state that is different from the first movement state is detected. The generation method includes: determining an exercise type carried out by the user, based on the body motion signal; acquiring the biological information in the first movement state, if an exercise of the exercise type is continued for a third period or longer and the first movement state is detected based on the body motion signal; and generating relation information between the biological information that is acquired and the exercise type that is determined.

According to this application example, the notification of the notification information is stopped if the movement state that is different from the first movement state is detected. Therefore, the consumption of driving electricity that is needed to notify the user of the notification information during this time can be restrained.

Also, if the first movement state is detected, the user is notified of the notification information. Therefore, the user can recognize the notification information in the first movement state. For example, if the first movement state is defined as the state where the user is not exercising, and the state that is different from the first movement state is defined as the state where the user is exercising, the user in the first movement state is not concentrating on the exercise and therefore is highly likely to notice the notification information and the information can easily communicated to the user. Meanwhile, if the movement state that is different from the first movement state is detected, electricity consumption is restrained. Therefore, based on the exercise state of the user, information measured during the exercise can be properly communicated to the user and electricity consumption can be restrained.

### BRIEF DESCRIPTION OF THE DRAWINGS

The invention will be described with reference to the accompanying drawings, wherein like numbers reference like elements.

FIG. 1 is an explanatory view showing an outline of a wearing device.

FIG. 2 is a sequence chart explaining display control processing of the wearing device.

FIG. 3 is a sequence chart explaining learning processing of the wearing device.

FIG. 4 is a block diagram showing a schematic configuration of the wearing device.

FIG. 5 is a flowchart showing the flow of the display control processing.

FIG. 6 is a flowchart showing the flow of summary information display processing.

FIG. 7 is a flowchart showing the flow of the learning processing.

FIG. 8 is a graph showing acceleration data during swimming.

FIG. 9 shows an example of exercise analysis data.

FIG. 10 is a graph showing changes in stroke pitch and pulse rate with time during a swimming movement of a user.

FIG. 11 shows an example of a learning table.

FIG. 12 shows an example of a display screen.

FIG. 13 shows an example of the screen of the summary information.

FIG. 14 shows an example of the screen of the summary information.

### DESCRIPTION OF EXEMPLARY EMBODIMENTS

Hereinafter, embodiments of the invention will be described with reference to the drawings. In the drawings below, respective parts and screens are shown with different scales and arrangement positions from the actual scales and arrangement positions in order to show these parts and screens in recognizable sizes.

### Embodiment 1

### Outline of Wearing Device

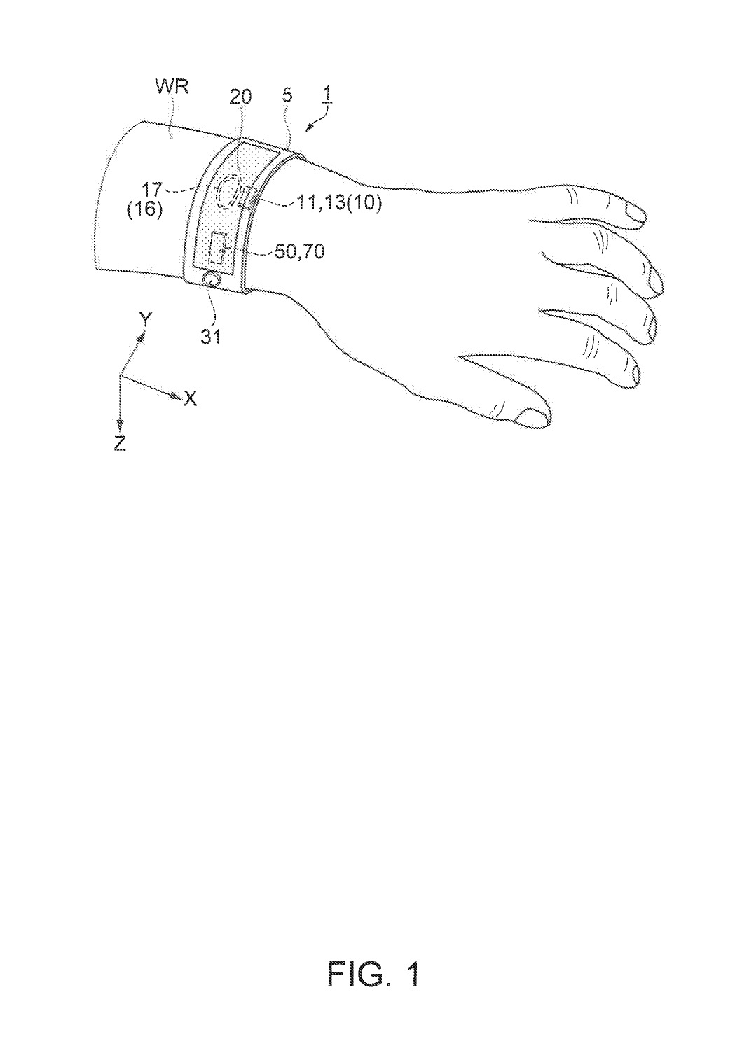

FIG. 1 is an explanatory view of a wearing device. In the description of this embodiment, an exercise based on the front crawl is employed as an example of exercise in the water such as swimming. However, as an exercise in the water, the breaststroke, the backstroke, the butterfly stroke, water walking, or water jogging may be employed as well. Also, the swimming movement in the description below includes a water walking movement and a water jogging movement.

A wearing device 1 shown in FIG. 1 is equivalent to a wearable device and is a wristwatch-type device worn with a strap 5 around a wrist WR (or an arm part including the upper arm) of a user exercising in the water. The wearing device 1 includes a pulse sensor 17 which is arranged, exposed on the surface on the wrist WR side of the strap 5, a display unit 20 which is arranged, exposed on the surface opposite to the wrist WR side of the strap 5, and an acceleration sensor 11, a pressure sensor 13, a processing unit 50, a storage unit 70 and a battery 31 or the like which are built inside the strap 5.

The acceleration sensor 11 and the pressure sensor 13 are included in a body motion detection unit 10 (body motion detector) and detect a body motion signal of the user. The acceleration sensor 11 detects an acceleration signal generated through movements of the wrist WR in the illustrated three axial directions (X-axis, Y-axis, and Z-axis). The pressure sensor 13 detects the entry into the water and the exit from the water of the wearing device 1 worn by the user. The pulse sensor 17 is included in a biological information detection unit 16 (biological information detector) and made up of a photoelectric sensor or the like. The pulse sensor 17 casts light on the wrist WR, receives the light reflected by a blood vessel in the wrist WR as pulsation, and calculates (detects) the pulse rate as biological information.

The processing unit 50 (processor) is a computer including a CPU or the like. The storage unit 70 is a memory such as a RAM or ROM. The processing unit 50 has a functional unit which is realized based on a program stored in the storage unit 70. The battery 31 is a primary battery, secondary battery or the like, and supplies electricity to each of the above-described parts under the control of the functional unit of the processing unit 50 (hereinafter simply referred to as the functional unit). The display unit 20 includes a display drive circuit and a display panel (neither of which are illustrated) or the like, and is switched between a display-on state and a display-off state under the control of the functional unit. When the functional unit switches the display unit 20 to the display-on state, the functional unit causes the battery 31 to supply electricity to the display drive circuit and the display panel or the like of the display unit 20 and thus causes the display unit 20 to display generated display information. Meanwhile, when the display unit 20 is switched to the display-off state by the functional unit, the supply of electricity from the battery 31 is cut off or the amount of electricity supplied is made very small and therefore the display on the display panel disappears.

The display unit 20 is equivalent to a notification unit. The display information is equivalent to notification information. The processing in which the functional unit of the processing unit 50 causes the display unit 20 to display generated display information is equivalent to processing in which the notification unit is made to notify the user of the notification information. The processing in which the display unit 20 is switched to the display-off state by the functional unit is equivalent to processing in which the notification of the notification information by the notification unit is stopped.

### Outline of Display Control Processing

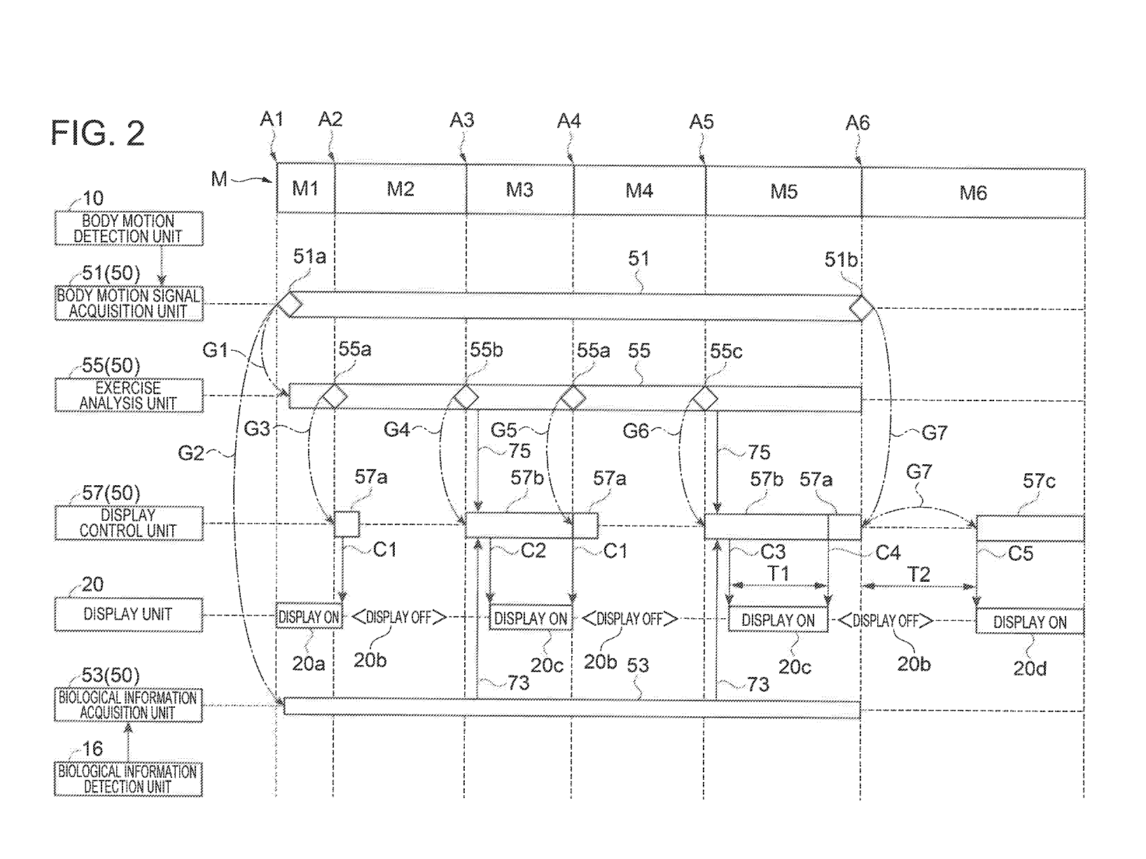

An outline of display control processing will be described with reference to FIG. 2. FIG. 2 is a sequence chart explaining the display control processing of the wearing device. A movement state M shown at the top of FIG. 2 shows changes of the movement state of the user in the shape of a strip. The movement state M shows that the movement state of the user changes along the passage of time (from left to right). The movement state M includes different movement states M1 to M6. Actions of the user that trigger these movement states are shown as actions A1 to A6, respectively. The actions A1 to A6 and the movement states M1 to M6 shown in FIG. 2 are defined as follows.

Action A1: entry into the water
Movement state M1: preparation action
Action A2: starting the swimming movement
Movement state M2: one-way outbound 25-m front crawl movement
Action A3: starting the turn movement
Movement state M3: turn movement
Action A4: starting the swimming movement
Movement state M4: one-way inbound 25-m front crawl movement
Action A5: ending the swimming movement and starting the static movement
Movement state M5: static movement
Action A6: exit from the water
Movement state M6: continuing the exit-from-water state

Here, the explanation of the outline of the display control processing focuses on the display state of the display unit 20 corresponding to the movement state M. The control processing on the display state of the display unit 20 (display control processing) is realized by a display control unit 57 of the processing unit 50. Details of the other functional units shown in FIG. 2 will be described later.

During the period of the movement state M1, the display unit 20 performs display (display-on 20a). In the display-on 20a, information about the detection state of signals measured during a swimming movement (whether signals detected by the acceleration sensor 11 and the pulse sensor 17 or the like are normally detected or not) is displayed.

When the movement state M, triggered by the action A2, shifts to the movement state M2, the display unit 20 is switched to the display-off state (display-off 20b).

When the movement state M, triggered by the action A3, shifts to the movement state M3, the display unit 20 performs display (display-on 20c). In the display-on 20c, display information (display screen) generated based on signals detected by the acceleration sensor 11 and the pulse sensor 17 or by one of these sensors during the period of the movement state M2 (one-way outbound) is displayed.

When the movement state M, triggered by the action A4, shifts to the movement state M4, the display unit 20 is switched to the display-off state (display-off 20b).

When the movement state M, triggered by the action A5, shifts to the movement state M5, the display unit 20 performs display (display-on 20c). In the display-on 20c, display information (display screen) generated based on signals detected by the acceleration sensor 11 and the pulse sensor 17 or by one of these sensors during the period of the movement state M4 (one-way inbound) is displayed for a period T1. After the lapse of the period T1, the display unit 20 is switched to the display-off state (display-off 20b). A screen D10 shown in FIG. 12 is an example of the display screen.

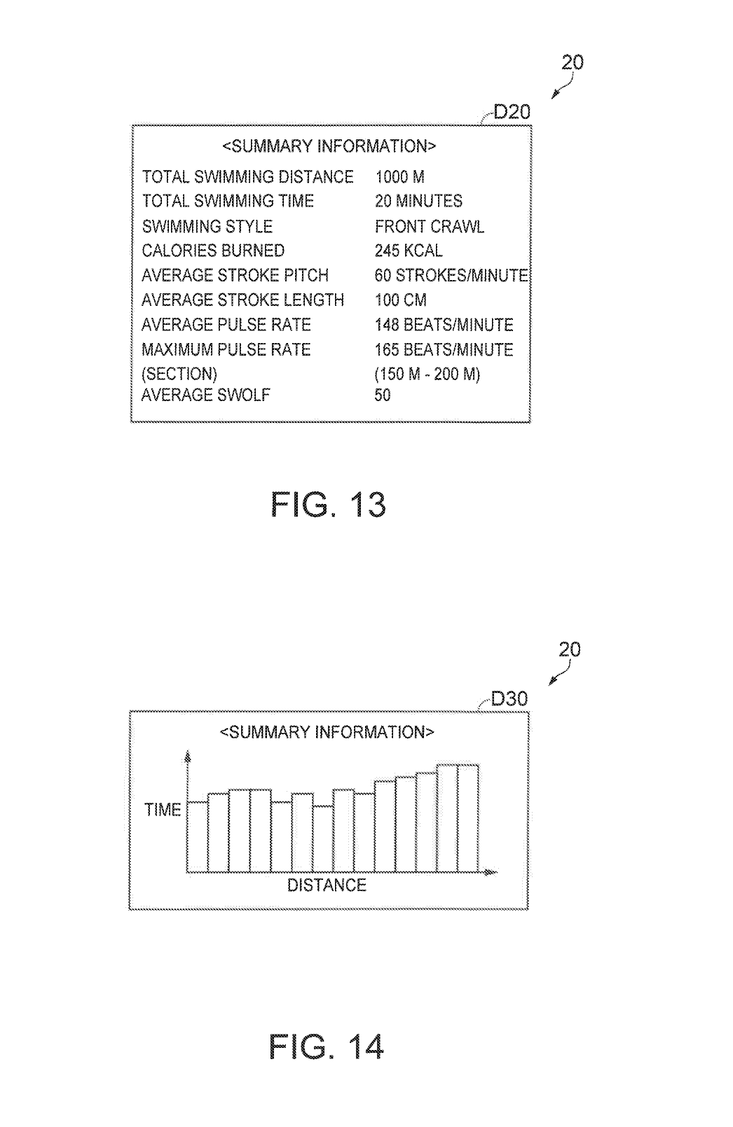

When the movement state M, triggered by the action A6, shifts to the movement state M6, a period T2 is timed. After the lapse of the period T2, the display unit 20 displays summary information (display-on 20d). The summary information includes analysis information about the movement of swimming after the entry into the water (movement state M2 and movement state M4). A screen D20 shown in FIG. 13 is an example of the screen of the summary information.

The period T1 is equivalent to a first period. The period T2 is equivalent to a second period.

In this way, in the wearing device 1, the display control processing by the processing unit 50 controls the display unit 20 in such a way that the display unit 20 is in the display-off state during the period of the swimming movement of the user (movement state M2 and movement state M4), whereas display information (display screen) is displayed on the display unit 20 during the period of the turn movement and static movement of the user (movement state M3 and movement state M5). That is, in the wearing device 1, during the period when the user is concentrating on the swimming movement and therefore cannot visually recognize the display unit 20, there is no significant effect even if the display unit 20 is switched to the display-off state of the display unit 20. Therefore, the display unit 20 is switched to the display-off state, thus restraining the driving electricity. Also, in the wearing device 1, information about a one-way swimming movement is displayed as display information at the timing of a turn movement or static movement when the user can visually recognize the display unit 20.

The movement state M3 and the movement state M5 are equivalent to a first movement state. The movement state M2 and the movement state M4 are equivalent to a second movement state.

### Outline of Learning Processing

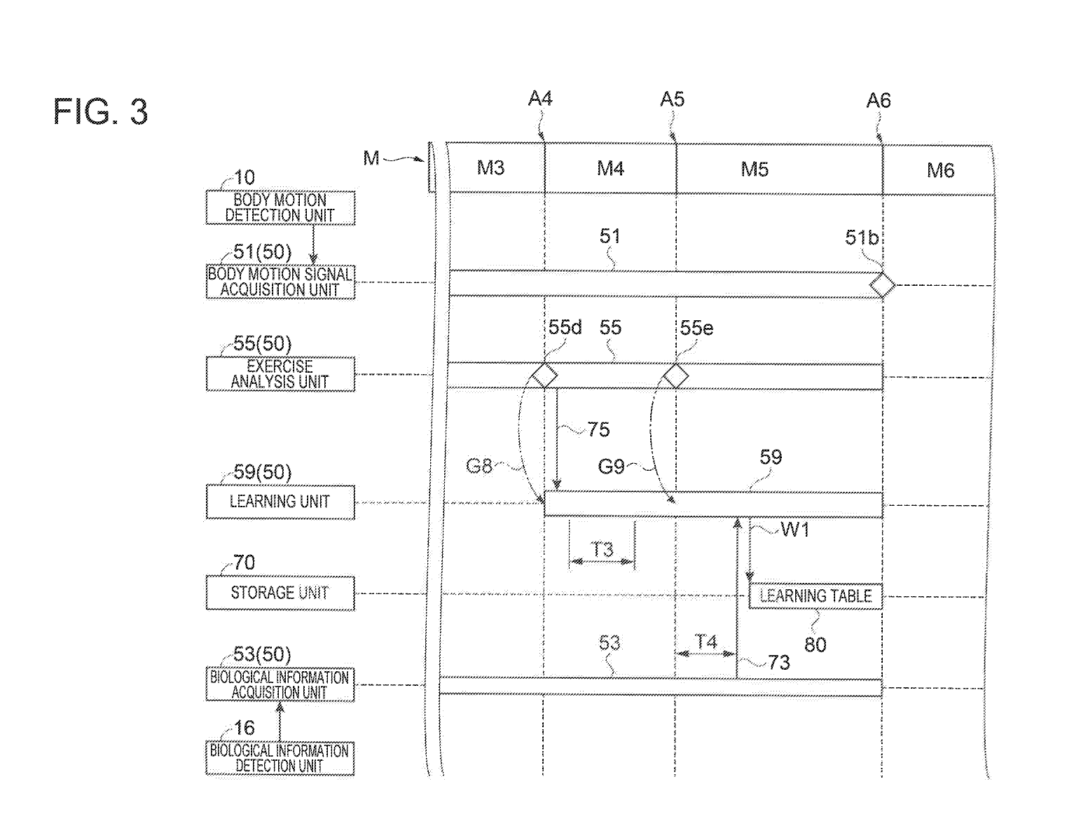

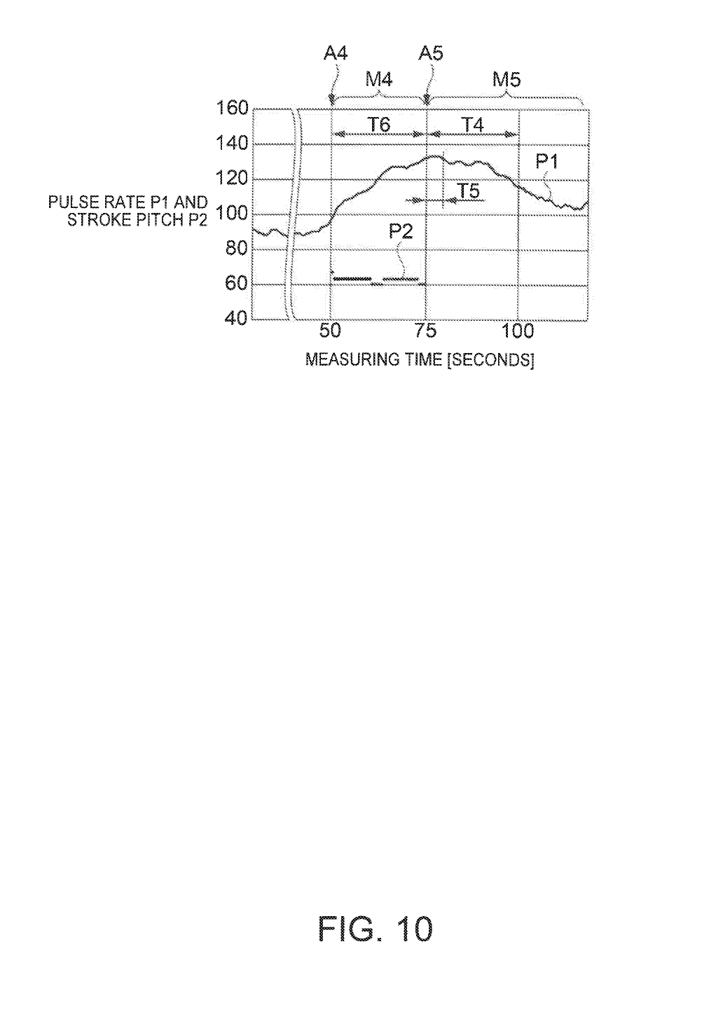

An outline of learning processing will be described with reference to FIGS. 3 and 10. FIG. 3 is a sequence chart explaining the learning processing of the wearing device. FIG. 10 is a graph showing changes in the stroke pitch and pulse rate with time during a swimming movement of the user. The swimming movement of the user shown in FIG. 10 is a specific example of the front crawl.

In the learning processing, a learning table 80 in which the exercise type of the exercise that is being carried out and the biological information acquired during or after the exercise are related to each other is stored in the storage unit 70. The learning table 80 is equivalent to relation information and is updated based on newly acquired exercise type and the corresponding biological information every time an exercise is carried out. The learning processing is equivalent to processing in which relation information between the exercise type and the acquired biological information is generated. The exercise type includes information about the swimming style such as the front crawl and the type of water exercise, and information about the exercise intensity such as stroke pitch. The biological information is the pulse rate detected by the biological information detection unit 16 and stored as biological information data 73. In the learning processing, if the biological information is successfully detected during the exercise, the biological information and the exercise type at the timing when the biological information is detected are stored in association with each other in the learning table 80. However, in the case of an exercise in the water like an exercise to be a measuring target of the wearing device 1, it is often difficult to detect the biological information data 73. More specifically, in the case of an exercise in the water, the user often makes complex movements with intense body motions with the upper limbs, compared with an exercise like running on the ground. In such cases, if body motions are reflected on the pulsation, the waveform signal outputted from the pulse sensor 17 may include many noises other than the pulse wave signal and therefore effective biological information data 73 may not be outputted. Particularly, during an exercise in the water, various impacts apply due to the rotating and twisting of the wrist WR around which the wearing device 1 is worn and the entry of the wrist WR into the water surface. Therefore, the pulse rate during the swimming movement may not be detected by the pulse sensor 17, resulting in missing data of the biological information data 73.

In the learning processing, the pulse rate is detected by the pulse sensor 17 at the time of a static movement after a swimming movement is finished, in case the pulse rate cannot be outputted during the swimming movement. This is to estimate the pulse rate in the swimming movement that is immediately before or the like, because the pulse rate does not immediately drop after an exercise like a swimming movement is suddenly finished (the sigmoid (S-shaped) change with time; see JP-A-2012-232010). As seen in the graph of FIG. 10, the pulse rate P1 is approximately 90 beats/minute before the action A4 and then rises to about 95 to 130 beats/minute during the period T6 in the movement state M4. The stroke pitch P2 represents the number of strokes taken per minute with the front crawl. The stroke pitch P2 is 60 to 65 strokes/minute during the period T6 in the movement state M4 and does not appear during the other periods, thus indicating that no stroke is taken (the user does not swim). With the action A5, the swimming movement is finished and the static movement is started. During the period T4 in the movement state M5 (static movement), the pulse rate P1 drops from 130 beats/minute to 115 beats/minutes. During the period T5 in the period T4, the pulse rate, which is stable substantially at the same level as in the last stage of the period T6, is detected.

The pulse rate data in the period T5, thus acquired, is stored in association with the exercise type of the swimming movement in the period T6 (front crawl, stroke pitch P2) in the learning table 80. The pulse rate data stored in the learning table 80 at this point is stored as a target pulse rate.

### Use of Learning Table

The learning table 80 updated by the learning processing is used to calculate the pulse rate in the swimming movement. The calculated pulse rate is displayed as information of the average pulse rate and the maximum pulse rate during the one-way swimming movement in the movement state M2 or the movement state M4 or the like in the display control processing, at the time of the turn movement or static movement in the movement state M3 or the movement state M5 or the like (see the screen D10 in FIG. 12).

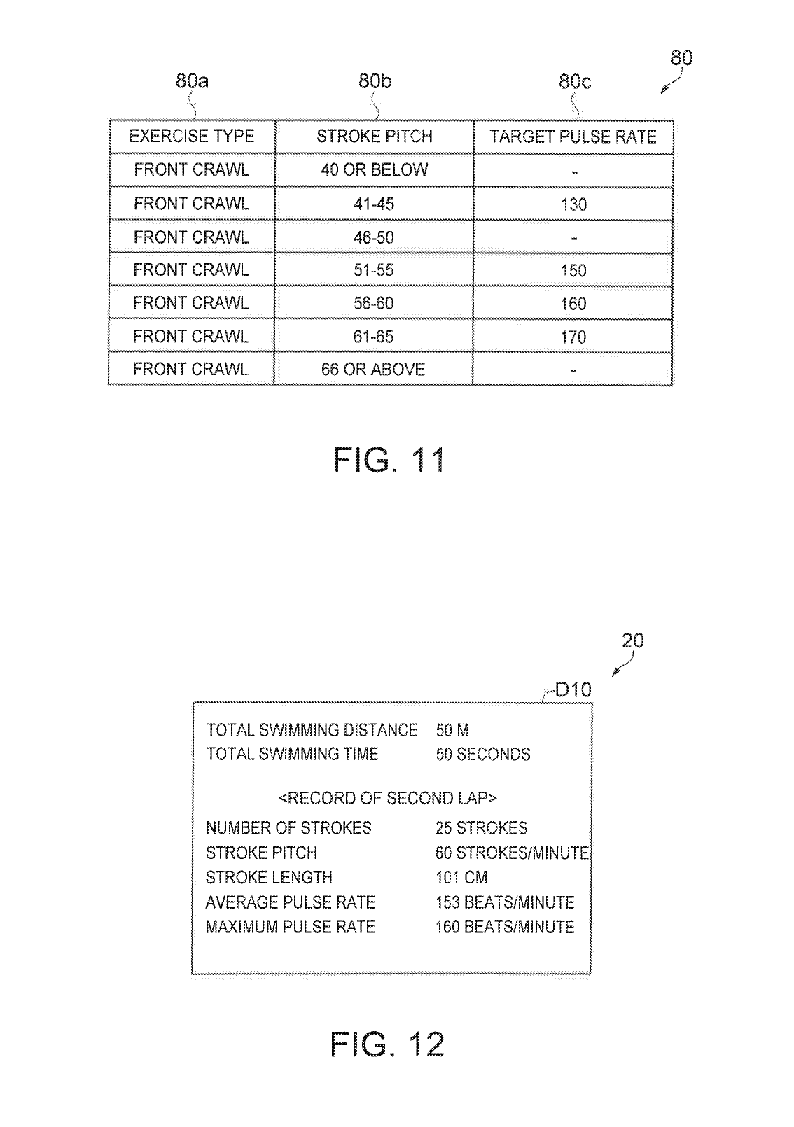

FIG. 11 illustrates an example of the learning table. The learning table 80 includes the exercise type (column 80a), the stroke pitch (column 80b), the target pulse rate (column 80c) or the like. The target pulse rate is information used to estimate the pulse rate, based on the information of the content of the exercise type and the value of the stroke pitch.

For example, in the display control processing, the pulse rate corresponding to the missing data of the biological information data 73 during the swimming movement is estimated, and the average pulse rate, the maximum pulse rate and the like which are necessary as display information are calculated. In the display control processing, the exercise type and the stroke pitch in the time bracket corresponding to the missing data included in the biological information data 73 are acquired from exercise analysis data 75. The target pulse rate is then acquired, based on the content of the exercise type and the value of the stroke pitch which are acquired, with reference to the learning table 80. In the display control processing, the pulse rate corresponding to the missing data is estimated using the target pulse rate. Then, the average pulse rate and the maximum pulse rate during the one-way swimming movement are calculated using the estimated pulse rate. The display information is thus generated and then outputted to the display unit 20.

In this way, in the learning processing, even if the pulse rate cannot be detected during the swimming movement, the pulse rate detected during the static movement immediately after the swimming movement is used as the target pulse rate in the learning table 80. In the display control processing, the missing pulse rate is estimated with reference to the learning table 80. The display information is generated based on the estimated pulse rate, at the timing of the turn movement or the static movement when the user can visually recognize the information. The display information can thus be displayed on the display unit 20.

The configuration and control method of the wearing device 1 to realize the display control processing and the learning processing will be described in detail below.

### Wearing Device

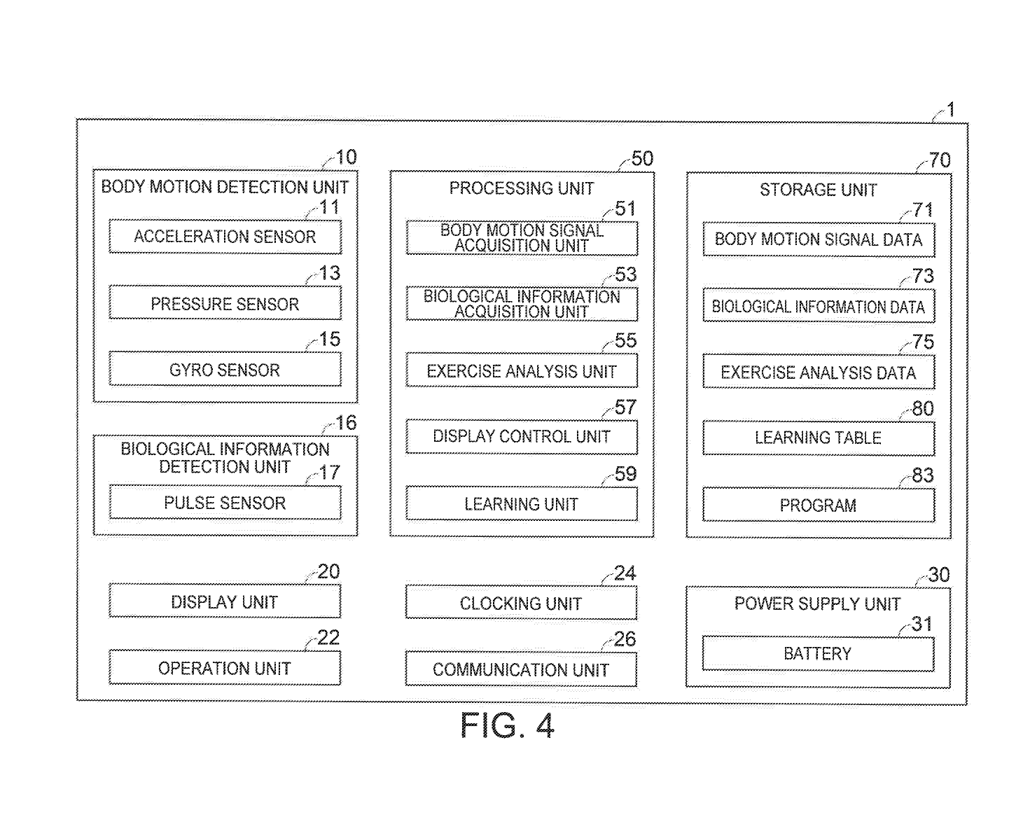

FIG. 4 is a block diagram showing a schematic configuration of the wearing device. The wearing device 1 includes the body motion detection unit 10, the biological information detection unit 16, the display unit 20, an operation unit 22, a clocking unit 24, a communication unit 26, a power supply unit 30, the processing unit 50, and the storage unit 70 or the like.

### Body Motion Detection Unit

The body motion detection unit 10 includes the acceleration sensor 11, the pressure sensor 13, and a gyro sensor 15 or the like.

The acceleration sensor 11 is a sensor which detects acceleration signals in the three axial directions of the X-axis, Y-axis, and Z-axis which are orthogonal to each other, as described above. The acceleration sensor 11 measures changes in acceleration on each axis at every sampling interval. As a preferable example, the sampling frequency is set at 16 Hz or above. The acceleration sensor 11 detects a movement of the user, then amplifies, shapes, and A/D-converts the detected acceleration signal by an amplifier circuit, a waveform shaping circuit, and an A/D conversion circuit (none of which is illustrated), and outputs the resulting signal as acceleration data to a body motion signal acquisition unit 51 (processing unit 50). The acceleration signal may be outputted to the biological information detection unit 16. In that case, the acceleration signal can also be used in restraining processing on a body motion noise superimposed on a pulse wave signal when the biological information detection unit 16 detects biological information. Also, the acceleration signal may be outputted to the operation unit 22 under the control of the processing unit 50. In that case, the operation unit 22 can acquire various user operations based on the acceleration signal.

The pressure sensor 13 is a sensor which detects a pressure signal every unit time. The pressure sensor 13 amplifies, shapes, and A/D-converts the pressure signal detected every unit time, by an amplifier circuit, a waveform shaping circuit, and an A/D conversion circuit (none of which is illustrated), and outputs the resulting signal as pressure data to the body motion signal acquisition unit 51 (processing unit 50). The pressure sensor 13 may also determine whether the value of the pressure data is of air pressure or water pressure, and outputs the result of the determination (for example, “0” for air pressure and “1” for water pressure) as pressure data to the body motion signal acquisition unit 51.

The gyro sensor 15 is a sensor which detects angular velocities about the three axes of the X-axis, Y-axis, and Z-axis. The gyro sensor 15 amplifies, shapes, and A/D-converts a gyro signal detected every unit time, by an amplifier circuit, a waveform shaping circuit, and an A/D conversion circuit (none of which is illustrated), and outputs the resulting signal as gyro data to the body motion signal acquisition unit 51 (processing unit 50). The processing unit 50 can calculate movements such as tilt and rotation of the wrist WR around which the wearing device 1 is worn.

The acceleration sensor 11 and the gyro sensor 15 use the three axial directions as detection targets. However, the use of the three axes is not limiting. One axis, two axes, or four or more axes may also be employed.

### Biological Information Detection Unit

The biological information detection unit 16 includes the pulse sensor 17.

The pulse sensor 17 is a sensor module which includes a photoelectric sensor and a computing circuit or the like and which detects the pulse wave of the user and calculates the pulse rate. The photoelectric sensor includes a light emitting element and a light receiving element. The photoelectric sensor casts light toward the wrist WR from the light emitting element and receives the reflected light reflected from a blood vessel, with the light receiving element. The pulse sensor 17 detects the pulse wave of the user, using a phenomenon in which the reflectance of light differs between the time of blood vessel expansion and the time of blood vessel contraction. The computing circuit performs frequency resolution processing on the data of the detected pulse wave, thus analyzes the signal intensity value of each frequency, and specifies a frequency spectrum corresponding to the pulse wave, based on a frequency spectrum including a noise as well as the pulse wave. The ratio of the signal of the pulse wave (S) to the noise (N), which is not the pulse wave, is referred to as the SN ratio and is used to determine the degree of reliability of the calculated pulse rate value. One of the causes of the noise, which is not the pulse wave, is the influence of the movement of the wrist WR in the swimming movement of the user. Therefore, the frequency spectrum corresponding to the pulse wave can be specified, referring to the acceleration signal, which changes with the movement of the wrist WR. However, the computing circuit tends to become unable to specify the frequency spectrum corresponding to the pulse wave if the movement of the wrist WR becomes intense.

The pulse sensor 17 outputs the calculated pulse rate to the processing unit 50. Also, the value of the SN ratio may be outputted to the processing unit 50.

The pulse sensor 17 may employ not only the photoelectric sensor but also an ultrasonic sensor which detects the contraction of the blood vessel with ultrasonic waves and thus measures the pulse rate, or a sphygmomanometer which detects the pulse pressure with a piezoelectric element or the like, and may also employ a sensor which supplies a weak current into the body from an electrode and thus measures the pulse rate.

### Display Unit

The display unit 20 is a display device capable of displaying letters and icons. The display unit 20 includes, for example, a flexible dot matrix EPD (electrophoretic display) which is flexibly deformable, and a display drive circuit or the like. The display drive circuit is switched between a driving-on state and a driving-off state in response to a signal outputted from the display control unit 57 of the processing unit 50. When the display drive circuit is driven, various kinds of display information outputted from the display control unit 57 are displayed.

The display unit 20 is not limited to the EPD and may also be an LCD (liquid crystal display), segment LCD, organic electroluminescence display or the like.

### Operation Unit

The operation unit 22 includes operation buttons or switches, or is a touch panel covering the display surface of the display unit 20 (none of which is illustrated), and outputs an operation signal corresponding to an operation by the user, to the processing unit 50. The operation unit 22 may also be configured as a functional unit which detects that a predetermined operation is carried out on the wearing device 1 intentionally by the user. The predetermined operation is, for example, the operation of tapping (lightly hitting) the surface of the wearing device 1 three times. If the operation unit 22 detects this operation, the operation unit 22 can switch the display content on the display unit 20. More specifically, the operation unit 22 can detect that the tapping is carried out three times consecutively, based on the pattern of the output signal of the acceleration signal outputted from the acceleration sensor 11. If the operation unit 22 detects that the tapping is carried out three times consecutively, the operation unit 22 realizes a predetermined function corresponding to that operation.

### Clocking Unit

The clocking unit 24 is a real-time clock and generates a sampling interval or a unit time to be used in the acceleration sensor 11, the pressure sensor 13, and the gyro sensor 15 or the like. The clocking unit 24 also has clocking functions such as a timer function, a calendar function, a clock function, and a stopwatch function.

### Communication Unit

The communication unit 26 is a short-range wireless adapter with its electricity consumption restrained, as a preferable example. The communication unit 26 transmits various data stored in the storage unit 70 and data such as the display information which the user is notified of, to another information device and a server or the like on a network. The communication unit 26 also receives a program or the like for realizing each functional unit of the wearing device 1 from another information device or the server or the like. The short-range wireless adapter may be, for example, a Bluetooth (trademark registered) adapter. However, this configuration is not limiting. Any communication adapter capable of wireless communication may be employed. A wireless LAN adapter having an IP (internet protocol) and a communication protocol shared with an external information terminal may be employed. The communication unit 26 may also include a physical communication terminal to connect to another information device or the like via a cable.

### Power Supply Unit

The power supply unit 30 includes the battery 31 and a power supply circuit or the like, and supplies electricity to each part of the wearing device 1 from the battery 31 under the control of the processing unit 50. If the battery 31 is a rechargeable secondary battery, the power supply unit 30 has a charger function for recharging the battery 31 with electricity supplied to the power supply circuit from outside in a contactless manner or via a charging terminal. The power supply unit 30 may also include a power generation unit (not illustrated). The power generation by the power generation unit in the wearing device 1 may be realized by a photovoltaic power generation (solar cells) or may be realized by vibration power generation, manual power generation, or temperature difference power generation or the like.

### Processing Unit and Storage Unit

The processing unit 50 is a control device and computing device which has a processor such as a CPU or DSP (digital signal processor) and comprehensively controls each part of the wearing device 1. The processing unit 50 includes functional units such as the body motion signal acquisition unit 51, a biological information acquisition unit 53, an exercise analysis unit 55, the display control unit 57, and a learning unit 59. These functional units are simply an example and not necessarily essential components. It can also be said that these functional units are functions or processing executed by the processing unit 50. Other functional units may also be included.

The storage unit 70 is made up of a storage device such as a ROM, flash ROM, RAM, HDD, or SSD. Body motion signal data 71, the biological information data 73, the exercise analysis data 75, the learning table 80, a program 83 and the like are stored in the storage unit 70.

Each functional unit of the processing unit 50 and each data stored in the storage unit 70 will be described below.

### Body Motion Signal Acquisition Unit

The body motion signal acquisition unit 51 acquires data related to the body motion signal such as acceleration data, pressure data, and gyro data from the body motion detection unit 10. Each of the acquired data is stored in time series as the body motion signal data 71 in the storage unit 70. The body motion signal acquisition unit 51 detects an entry into the water and an exit from the water, based on the pressure data, and the processing of the functional units of the exercise analysis unit 55 and the biological information acquisition unit 53 is started. Details will be described referring to FIG. 2.

When the body motion signal acquisition unit 51 has acquired pressure data from the body motion detection unit 10, the body motion signal acquisition unit 51 determines whether the device has entered the water or not, based on the amount of change of the pressure data (whether there is a change from the air pressure value to the water pressure value or not) (determination 51a). If the entry into the water is determined by the determination 51a, the processing of the exercise analysis unit 55 is started (execution G1) and the processing of the biological information acquisition unit 53 is started (execution G2). The execution G1, the execution G2, and executions G3 to G9, described below, express that the programs of the target functional units are executed by the CPU. When executed, these programs are executed in parallel with other programs by parallel processing or pseudo-parallel processing. Between the programs that are being executed, various data can be transmitted and received bidirectionally via inter-process communication or a semaphore or the like.

The body motion signal acquisition unit 51 determines whether the device has exit the water or not, based on the amount of change of the pressure data (whether there is a change from the water pressure value to the air pressure value or not) (determination 51b). If the exit from the water is determined by the determination 51b, the timer function of the clocking unit 24 is started and the start of the processing (execution G7) of the display control unit 57 (summary display processing 57c) after the lapse of the period T2 is set.

If the exit from the water is determined by the determination 51b, the body motion signal acquisition unit 51 notifies the exercise analysis unit 55, the learning unit 59, and the biological information acquisition unit 53 that the exit from the water is determined.

### Biological Information Acquisition Unit

The biological information acquisition unit 53 acquires the biological information such as the pulse rate detected or calculated by the biological information detection unit 16 and the relation information such as the SN ratio of the pulse rate. Each of the acquired data is stored in time series as the biological information data 73 in the storage unit 70.

### Exercise Analysis Unit

The exercise analysis unit 55 analyzes the movement of the user, based on the data related to the body motion signal. The processing of the display control unit 57 and the learning unit 59 is started according to the type of the analyzed movement. More specifically, the exercise analysis unit 55 analyzes the body motion signal such as the acceleration data stored in time series, referring to the body motion signal data 71, and determines the action and movement state of the user.

The movement state of the user to be analyzed is roughly categorized into a second movement state where the user is concentrating on an exercise and a first movement state where an exercise is paused (the user is not concentrating on an exercise).

The second movement state may be a movement state where the user is carrying out an exercise such as water waling or water aerobics, in addition to the swimming movement as in the movement state M2 and the movement state M4. The user in a movement state like this is concentrating on the exercise and therefore it is difficult for the user to visually recognize the display unit 20 of the wearing device 1. The swimming movement may be the front crawl, the breaststroke, the backstroke, the butterfly stroke or the like. The exercise analysis unit 55 analyzes these types of swimming styles. For each swimming movement, the exercise analysis unit 55 calculates information about the content of the swimming movement such as the number of strokes taken, time, and speed over a one-way distance (for example, 25 m), the stroke pitch, which is the number of strokes taken per minute, and the stroke length, which is the traveling distance per stroke. The information about the content of the swimming movement is stored as the exercise analysis data 75 every predetermined unit time (for example, every second).

The first movement state is a movement state where the continuation of the exercise in the second movement state is temporarily interrupted (paused), such as the turn movement as in the movement state M3 and the movement state M5. In a movement state like this, the user can visually recognize the display unit 20 of the wearing device 1 during the pause in the exercise, when the user is not concentrating on the exercise.

The determination on the movement state of the user as the first movement state is equivalent to detecting the first movement state. Similarly, the determination on the movement state of the user as the second movement state is equivalent to detecting the second movement state.

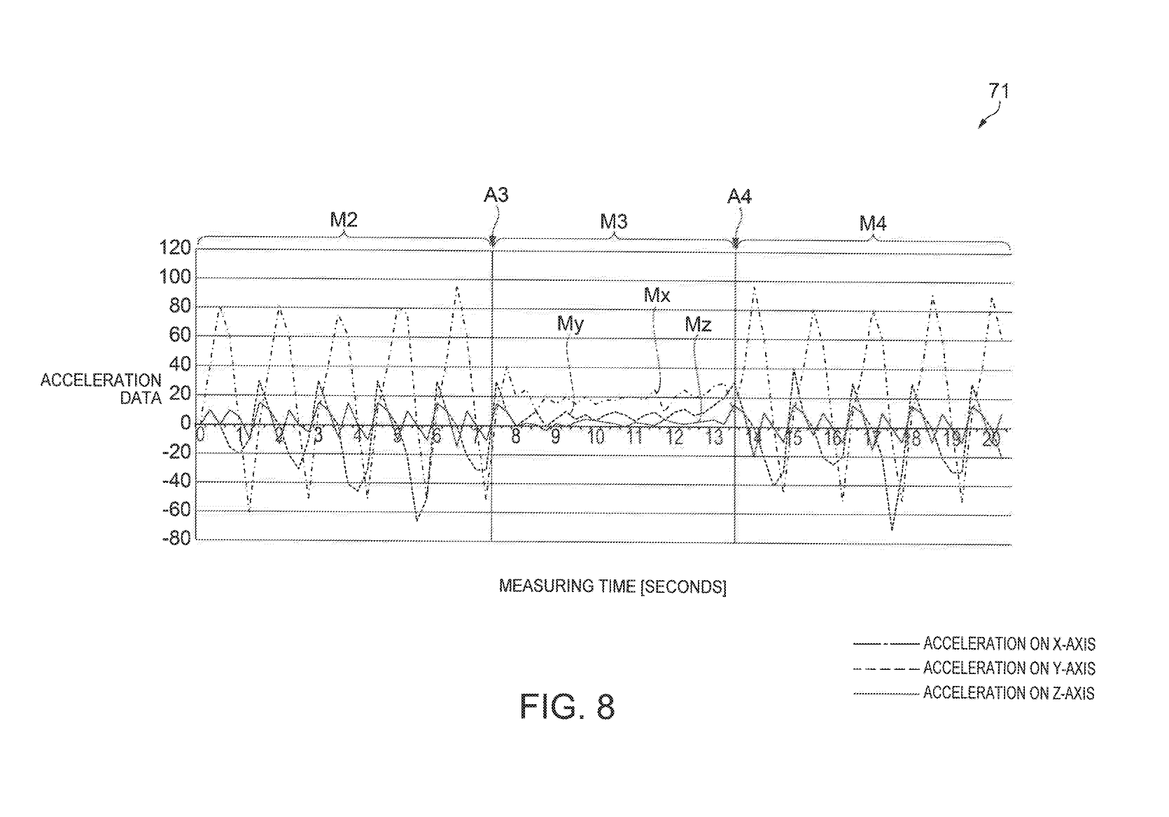

Next, the processing in which the exercise analysis unit 55 analyzes the action and movement state of the user, based on the body motion signal, will be described. FIG. 8 is a graph showing acceleration data during swimming.

This graph shows an example of changes of acceleration data in three axial directions when the user is swimming in the front crawl. The vertical axis of the graph represents the range of values of A/D-converted acceleration signal, and in this example, the range of −80 to +120. The horizontal axis represents the measuring time, and the numerical values on the scale represent the time elapsed in seconds with the left end being “0”. In the direction of the horizontal axis, it is shown that the movement state M2, the action A3, the movement state M3, the action A4, and the movement state M4 defined in the explanation with reference to FIG. 2 are carried out in their respective corresponding time brackets.

A graph line Mx shows acceleration data in the direction of the X-axis. A graph line My shows acceleration data in the direction of the Y-axis. A graph line Mz shows acceleration data in the direction of the Z-axis.

In the movement state M2, with the rotations of the arm in the front crawl, the graph line Mx shows that acceleration appears periodically with a peak at approximately “80” in the positive direction on the X-axis, which is the direction in which the user extends the arms in the traveling direction, and with a peak at approximately “−60” in the negative direction on the X-axis. The graph line My shows that acceleration appears in a periodic manner similar to the graph line Mx, with a peak at approximately “30” in the positive direction on the Y-axis, which is the direction in which the arms move away from the trunk, and with a peak at approximately “−20 to −60” in the negative direction on the Y-axis, which is the direction in which the arms move toward the trunk. The graph line Mz shows small fluctuations in the positive direction on the Z-axis, which is the direction of the depth of water, and in the negative direction, which is the opposite direction, with a period approximately half the period of the graph lines Mx and My.

In the movement state M3, which represents the movement state of the arms during a turn movement, all of the graph lines Mx, My, and Mz have small values and show no prominent periodicity, unlike in the movement state M2.

In the movement state M4, all of the graph lines Mx, My, and Mz show trends substantially similar to those in the movement state M2.

The exercise analysis unit 55 manages an action determination table (not illustrated) where patterns, characteristics, feature points and the like of acceleration data corresponding to actions and movement states of the user are stored. The action determination table is stored in the storage unit 70 in advance. The exercise analysis unit 55 reads data corresponding to a predetermined period (for example, the most recent four seconds), of the body motion signal data 71 stored successively with the movement of the user, every predetermined time (for example, every second), and outputs the corresponding action and movement state of the user with reference to the action determination table. In this way, the exercise analysis unit 55 can analyze the movement of the user almost in real time in response to changes in the action and movement state of the user.

The periods of the movement state M2 and the movement state M4 shown in FIG. 8 are characterized in that the acceleration data in the three axial directions show periodicity during these periods. Based on this characteristic, the exercise analysis unit 55 can determine that the movement state is a swimming movement or water walking with periodicity. The exercise analysis unit 55 also determines that the user is doing the front crawl, based on the respective waveform trends of the graph lines Mx, My, and Mz. The determination on the swimming style like this is also disclosed, for example, in publicly known literature (U.S. Pat. No. 8,652,010). In the movement state M3 between the action A3 and the action A4, the accelerations in the three axial directions no longer show periodicity and the total sum of the magnitudes of acceleration in the three axial directions is lower. If the magnitude of acceleration is equal to or below a predetermined threshold (for example, approximately one-fifth of the magnitude of acceleration at the time of the swimming movement), the exercise analysis unit 55 determines that the movement is a slow turn movement or a static movement. The method for determining the turn movement is also disclosed in publicly known literature (JP-A-2008-253470). If the acceleration data in the three axial direction show no periodicity and the magnitude of acceleration is low (than the predetermined threshold), the movement may be determined as a static movement.

If the acceleration data in the three axial directions show no periodicity but the magnitude of acceleration is higher (than the predetermined threshold) (i.e., the movement is intense), the exercise analysis unit 55 can determine that it is a movement state where the user is doing an exercise like water aerobics.

After determining the movement state, the exercise analysis unit 55 analyzes the exercise content in that movement state and stores the result of the analysis as the exercise analysis data 75 in the storage unit 70. As the exercise analysis data 75, information about the determined movement state and the content of the swimming movement at that time is stored in time series every predetermined time (for example, every second), as described above. For example, information of contents such as the front crawl and the stroke pitch is stored every second.

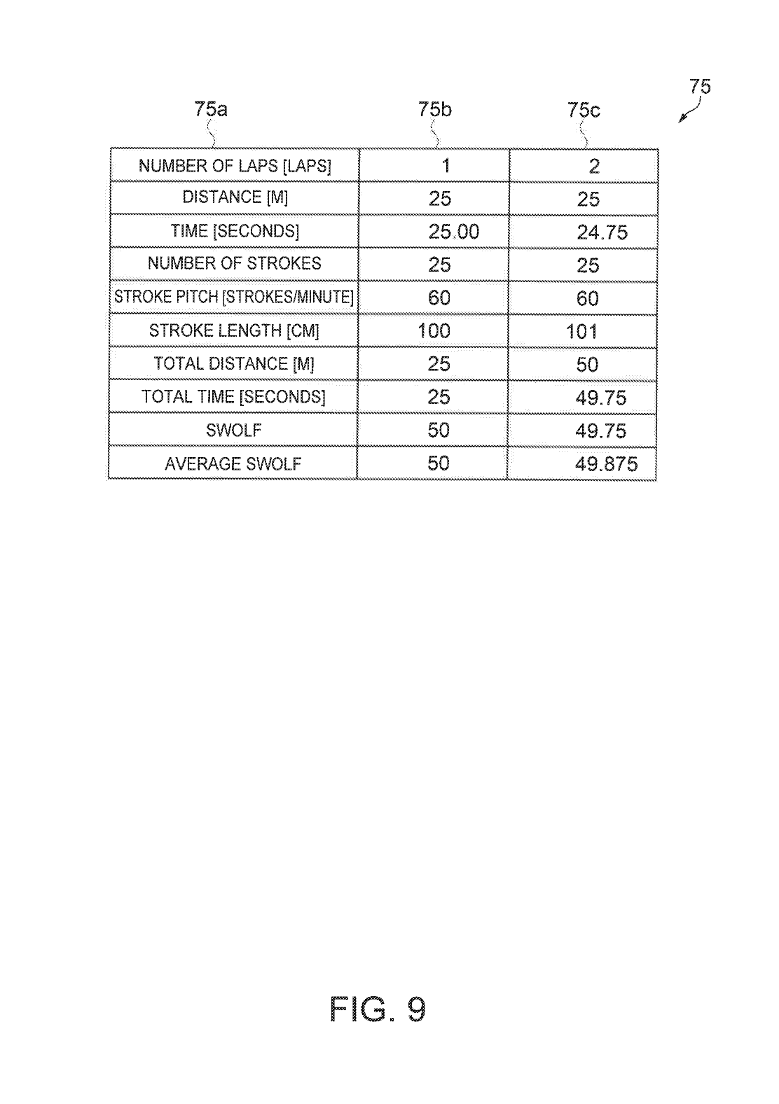

Also, information obtained by analyzing the exercise in each one-way swimming movement is stored as the exercise analysis data 75. FIG. 9 shows an example of this. FIG. 9 shows an example of the exercise analysis data. The exercise analysis data 75 shown in FIG. 9 represents information stored at the timing when a one-way swimming movement is finished. A column 75a show exercise content items to be analyzed. Columns 75b and 75c show exercise contents carried out by the user. The “number of laps [laps]” in the first row of the column 75a is the number of laps the user has swum one way in the swimming pool. In the respective item sections of the column 75b, the exercise contents of the first lap are stored. In the respective item sections of the column 75c, the exercise contents of the second lap are stored. Next, each item in the second row and onward will be described.

The “distance [m]” is the one-way distance of the swimming pool. This is a distance inputted in advance via the operation unit 22 of the wearing device 1.

The “time [seconds]” is the result of the time taken by swimming one way.

The “number of strokes” is the result of the number of strokes taken on one way.

The “stroke pitch [strokes/minute]” is the number of strokes taken per minute. A frequency spectrum is acquired by applying frequency resolution processing to the waveform data (FIG. 8) of acceleration, and the stroke pitch in the swimming movement is specified based on this frequency spectrum. Alternatively, the stroke pitch may be calculated using the number of peaks per unit time on a specified axis of acceleration data (for example, graph line Mx), instead of using frequency resolution processing.

The “stroke length [cm]” is the length traveled by one stroke. The stroke length is calculated using the values of the “stroke pitch” and the “distance”. Also, speed information may be calculated from the acceleration data in the traveling direction, and the traveling distance in the traveling direction per stroke may be calculated.

The “total distance [m]” is calculates by summing up the “distance [m]” on each one way.

The “total time [seconds]” is calculated by summing up the “time [seconds]” on each one way.

The “SWOLF” is the SWOLF score, which is calculated from the sum of the “time [seconds]” on one way and the number of strokes taken on one way.

The “average SWOLF” is the average value of the SWOLF scores on each one way.

As the exercise analysis unit 55 determines the action of the user, the processing of the display control unit 57 and the learning unit 59 corresponding to the action that is determined is started. The processing of the display control unit 57 and the learning unit 59 is executed in parallel with the exercise analysis unit 55.

As shown in FIG. 2, when the exercise analysis unit 55 determines the action A2 (start of the swimming movement) (determination 55a), the processing of display-off processing 57a included in the display control unit 57 is started (execution G3). When the exercise analysis unit 55 determines the action A3 (start of the turn movement) (determination 55b), the processing of display-on processing 57b of the display control unit 57 is started (execution G4). When the exercise analysis unit 55 determines the action A4 (start of the swimming movement) (determination 55a), the processing of the display-off processing 57a included in the display control unit 57 is started (execution G5). When the exercise analysis unit 55 determines the action A5 (end of the swimming movement and start of the static movement) (determination 55c), the processing of the display-on processing 57b is started (execution G6).

As shown in FIG. 3, when the exercise analysis unit 55 determines the action A4 (start of the swimming movement) (determination 55d), the processing of the learning unit 59 is started (execution G8). While the movement state M4 (swimming movement) of the user is continued after the determination on the action A4, the exercise analysis unit 55 analyzes the exercise content (that the movement state is a swimming movement). After determining the action A5 (end of the swimming movement and start of the static movement) (determination 55e), the exercise analysis unit 55 transmits information that the action A5 is determined, to the learning unit 59 (execution G9). While the movement state M5 (static movement) is continued after the determination on the action A5, the exercise analysis unit 55 analyzes that the movement state is a static movement.

### Display Control Unit

The display control unit 57 is a functional unit which realizes the display control processing. The display control unit 57 controls the display-on and display-off of the display unit 20. The display control unit 57 acquires or calculates information to be displayed on the display unit 20 and generates a display screen to display the information. The display control unit 57 subsequently drives the display drive circuit to output a display instruction command and the information of the display screen to the display unit 20. The display screen is displayed on the display unit 20. The display control unit 57 also switches the display drive circuit to a non-driving state and thus switches the display unit 20 to the display-off state. The information and display screen to be displayed on the display unit 20 are both equivalent to display information.

When the display control unit 57 is started based on the determination in the determination 55a by the exercise analysis unit 55 (execution G3), the display control unit 57 executes the display-off processing 57a and switches the display unit 20 to the display-off 20b (control C1).

When the display control unit 57 is started based on the determination in the determination 55b by the exercise analysis unit 55 (execution G4), the display control unit 57 executes the display-on processing 57b, reads necessary information for the display from the biological information data 73 and the exercise analysis data 75, and generates a display screen. The display control unit 57 then switches the display unit 20 to the display-on 20c and outputs the generated display screen to the display unit 20 (control C2).

When the display control unit 57 is started based on the determination in the determination 55a by the exercise analysis unit 55 (execution G5), the display control unit 57 executes the display-off processing 57a and switches the display unit 20 to the display-off 20c (control C1).

When the display control unit 57 is started based on the determination in the determination 55c by the exercise analysis unit 55 (execution G6), the display control unit 57 executes the display-on processing 57b, reads necessary information for the display from the biological information data 73 and the exercise analysis data 75, and generates a display screen. The display control unit 57 then switches the display unit 20 to the display-on 20c and outputs the generated display screen to the display unit 20 (control C3).

FIG. 12 shows an example of the display screen. A screen D10 includes the information read from the exercise analysis data 75 generated by the exercise analysis unit 55, the information read from the biological information data 73, and the information generated based on these pieces of information. The display contents of the screen D10 show the cumulative information about the movement state such as the total swimming distance (cumulative swimming distance) and the total swimming time. As the cumulative information, the cumulative number of turns, the cumulative number of strokes or the like may be displayed as well. As the record of the second lap (movement state M4), the number of strokes, the stroke pitch, and the stroke length are shown. The record of the second lap may also include the swimming time of the second lap. The “average pulse rate 153 beats/minute” and the “maximum pulse rate 160 beats/minute) in the screen D10 are generated based on the information read from the biological information data 73 or the pulse rate estimated by the display control unit 57 (details will be described later).

The screen D10 is an example of the display screen. The display screen may include at least one of the display contents and may also display a content other than the foregoing display contents. Also, the user may be enabled to select an arbitrary display content.

At the same as switching the display unit 20 to the display-on 20c by the control C3, the display control unit 57 starts the timer of the clocking unit 24 and thus times the period T1. As the period T1 passes, the display control unit 57 executes the display-off processing 57a and switches the display unit 20 to the display-off 20b by a control C4.

When the processing by the display control unit 57 is started (execution G7) after the lapse of the period T2 following the determination on the exit from the water in the determination 51b by the body motion signal acquisition unit 51, the display control unit 57 executes the summary display processing 57c. In the summary display processing 57c, analysis information about the swimming movement up to the exit from the water after the entry into the water (movement state M2 and movement state M4) or the like is included. Specifically, by a control C5, a screen of summary information is generated by gathering together the information of the display-on 20c displayed up to this point, and the display unit 20 is switched to the display-on 20d. FIG. 13 shows an example of the screen of the summary information. In the screen D20, the total swimming distance (swimming distance), total swimming time (swimming time), swimming style, calories burned, average stroke pitch, average stroke length, average pulse rate, maximum pulse rate, average SWOLF and its section, and the like are displayed, as the summary information. FIG. 14 shows an example of the screen of the summary information. In a screen D30, the swimming time in each section (one way) is shown in the form of a bar chart, as the summary information.

Also, the number of turns, degree-of-fatigue information, physical strength evaluation, training effect, and best time recorded over a one-way distance between turn movements, and the like may be included as the summary information. The number of turns, swimming style, and best time recorded over a one-way display between turn movements are derived by referring to the exercise analysis data 75. Details of the calories burned, degree-of-fatigue information, physical strength evaluation, and training effect will be described later as modification examples.

Next, details of the processing of generating display information by the display control unit 57 will be explained.

The display control unit 57 determines whether a pulse rate value with high reliability is success fully measured in the pulse rate data of the biological information data 73, or not. If a pulse rate with high reliability is successfully measured, the display control unit 57 generates display information based on the pulse rate data acquired from the biological information data 73. Meanwhile, if a pulse rate value indicating a partly unreliable value or abnormal value (prominent pulse rate value) exists in the biological information data 73 stored in time series, or if there is missing data that is not successfully measured, the display control unit 57 estimates pulse rate data which can substitute such pulse rate data, and thus corrects the pulse rate data. The method for determining whether a pulse rate value with high reliability is successfully measured or not can be realized, for example, by determining whether the value of the SN ratio outputted from the pulse sensor 17 is equal to or above a predetermined threshold, or not.

The processing of correcting a defective pulse rate value such as an unreliable pulse rate value, abnormal value, or missing data will be explained specifically. Information of the exercise type (including exercise intensity) corresponding to the time when the defective pulse rate value is generated is acquired with reference to the biological information data 73 and the exercise analysis data 75. Next, referring to the learning table 80, a target pulse rate corresponding to this exercise type is acquired.

To explain this using the example of the learning table 80 shown in FIG. 11, the display control unit 57 acquires the exercise type and the stroke pitch corresponding to the time when the defective pulse rate value is generated, and acquires a target pulse rate in the column 80c, referring to the column 80a (exercise type) and the column 80b (stroke pitch) of the learning table 80.

The display control unit 57 estimates a value of pulse rate corresponding to the pulse rate data having the defective pulse rate value, using the pulse rate value with high reliability existing before or after the defective pulse rate value with reference to the biological information data 73, and the acquired target pulse rate (see JP-A-2012-232010). The part of the pulse rate data having the defective pulse rate value of the biological information data 73 is replaced with the estimated value of pulse rate. Using the biological information data 73 thus updated, the average pulse rate and the maximum pulse rate are calculated. A display screen including the information of the calculated average pulse rate and maximum pulse rate is generated and then outputted to the display unit 20.

Learning Unit

The learning unit 59 is a functional unit which realizes the learning processing. The learning unit 59 generates the learning table 80. The learning unit 59 also updates the contents of the learning table 80. As shown in FIG. 3, when the start of the swimming movement is determined in the determination 55d by the exercise analysis unit 55, the processing of the learning unit 59 is started by the execution G8. After that, when the start of the static movement is determined in the determination 55e by the exercise analysis unit 55, information to that effect (start of the static movement) is transmitted to the learning unit 59, which is being executed (execution G9). Details of this will be described later.

By the execution G8, the learning unit 59 starts the timer function of the clocking unit 24 to start timing a period T3. The learning unit 59 acquires the exercise analysis data 75 in the movement state M4 and determines whether the period when the exercise content (information of the exercise type including exercise intensity) is stable is equal to or longer than the period T3, or not. In the determination on whether the exercise content is stable or not, as in the determination on the exercise type, the exercise content is determined as stable if the change in exercise intensity is within a predetermined range, whereas the exercise content is determined as not stable otherwise. If it is determined that the period when the exercise content is stable is equal to or longer than the period T3, a learning flag is switched ON (learning is possible). Otherwise, the learning flag is switched OFF (learning is impossible). The learning flag is stored in the storage unit 70 (not illustrated). The period T3 is equivalent to a third period.

By the execution G9, when a notification that the movement state is changed from the movement state M4 (swimming movement) to the movement state M5 (static movement) is given (transmitted) to the learning unit 59, the learning unit 59 acquires the content of the learning flag, and performs learning processing based on the information of the exercise analysis data 75 in the movement state M4 if the learning flag is ON.

First, the period when the exercise content in the movement state M4 is stable is extracted. This extraction period is the period when the exercise content is stable, going back from the endpoint of the swimming movement in the movement state M4 (determination 55e). This period has a length at least equal to or longer than the period T3.

After the extraction period is decided, the learning unit 59 determines whether biological information is stably detected during that period or not. Specifically, the SN ratio of the pulse rate of the biological information data 73 in the extraction period is evaluated, thus determining whether the degree of reliability of the pulse rate (value) is high or not.

If the learning unit 59 determines that the degree of reliability of the pulse rate is high, it means that the exercise content during the extraction period in the movement state M4 is stable and that biological information is stably detected. Therefore, the learning unit 59 stores the biological information (pulse rate) corresponding to the exercise content (exercise type and exercise intensity) into the learning table 80 (update W1).

If the learning unit 59 determines that the degree of reliability of the pulse rate (value) is not high, it means that the exercise content during the extraction period in the movement state M4 is stable but that biological information is not stably detected. In this case, the learning unit 59 starts the timer function of the clocking unit 24 to start timing the period T4 immediately after the execution G9. When the period T4 has passed, the learning unit 59 acquires the biological information (pulse rate) detected during that period, from the biological information data 73. During the period T4, the user is in the movement state M5 (static movement). Therefore, there are very few noises other than the pulse wave signal and the degree of reliability of the biological information data 73 is likely to be high. The exercise content (exercise type and exercise intensity) during the extraction period in the movement state M4 and the biological information (pulse rate) in the period T4 are stored in association with each other in the learning table 80 (update W1). The biological information during the exercise in the extraction period in the movement state M4 can be estimated based on the biological information during the period T4 in the movement state M5 after the end of the movement state M4. This utilizes the characteristic of the sigmoid change with time in pulse rate, explained in the outline of the learning processing.

The learning unit 59 may decide biological information excluding data with a relatively low degree of reliability, instead of referring to all the biological information data 73 during the period T4. The learning unit 59 may also select biological information corresponding to a period when the amount of change in pulse rate is small, at around the start of the period T4 or before the start of the period T4. For example, the period T5 shown in FIG. 10 is equivalent to the period when the amount of change is small. In this way, the learning unit 59 extracts a pulse rate with a high degree of reliability or a pulse rate during a period when the amount of change is small before or after the start of the period T4, and updates the learning table 80 by associating these pulse rates with the exercise type and exercise intensity of the immediately preceding exercising during the period T3 in the movement state M4. Thus, information with a higher degree of reliability can be provided.

The learning unit 59 stores the extracted pulse rate as a target pulse rate in the column 80c of the learning table 80 (FIG. 11), in association with the exercise type and exercise intensity (stroke pitch).

The values expressing the periods T3 and T4 are stored in the storage unit 70 in advance. As these values, different values are set depending on the exercise type and the one-way distance. For example, in the case of swimming one-way 25 m in the front crawl, the period T3 is set to be approximately 10 to 20 seconds, and the period T4 is set to be approximately 10 seconds.

### Flow of Control Method

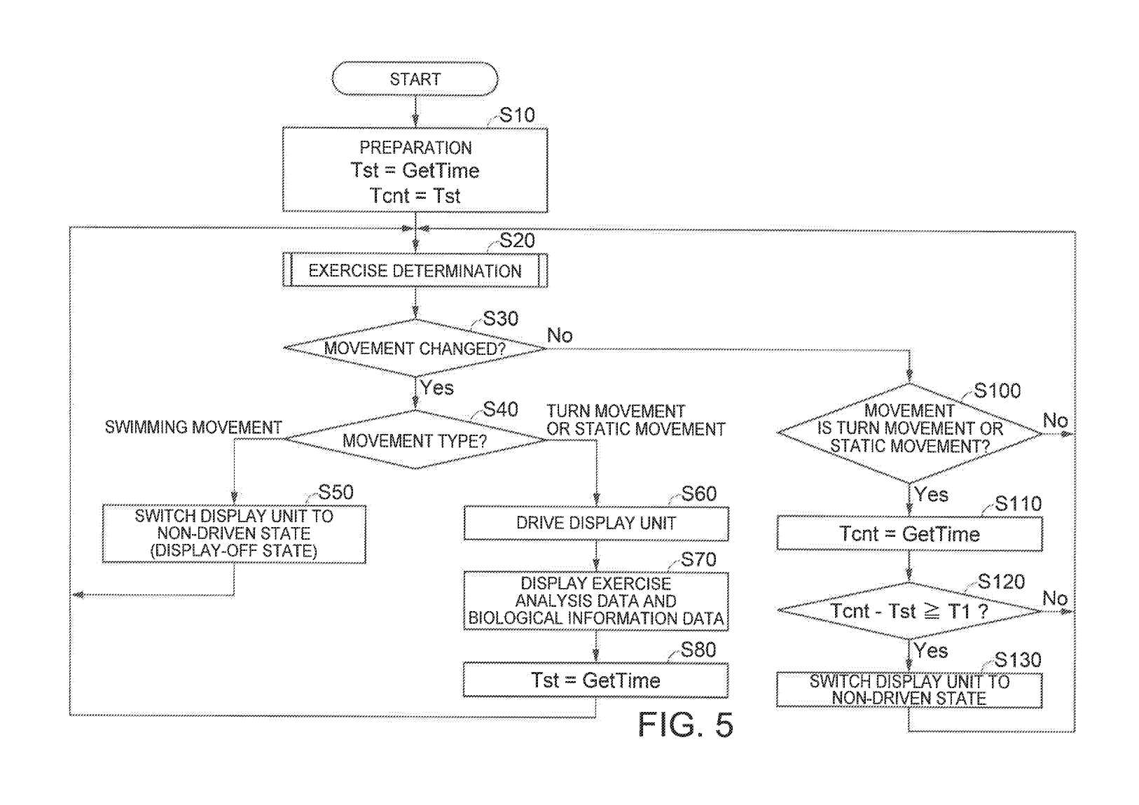

Next, the control method will be described, referring mainly to FIGS. 5, 6 and 7, and also to FIGS. 2 and 3 or the like when needed.

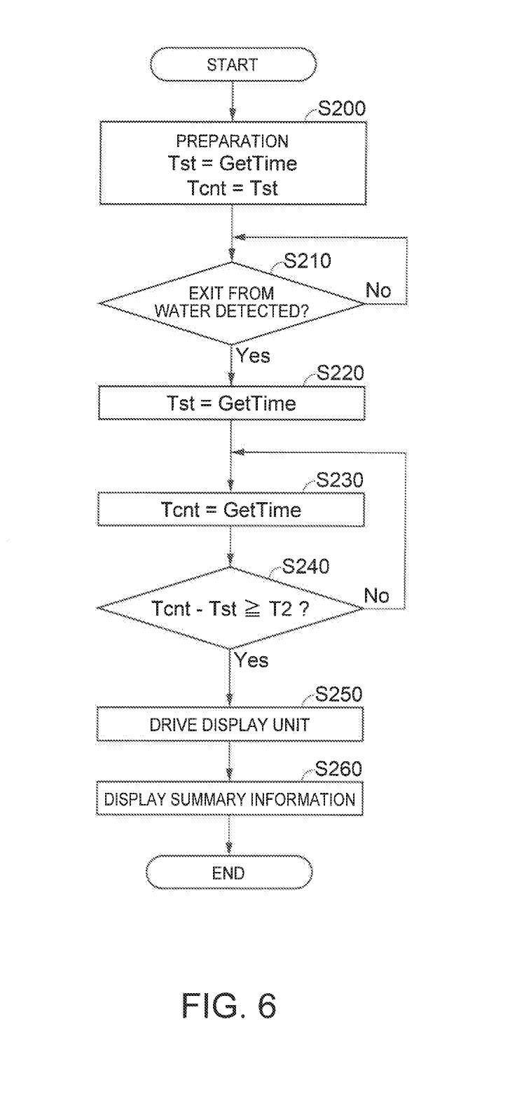

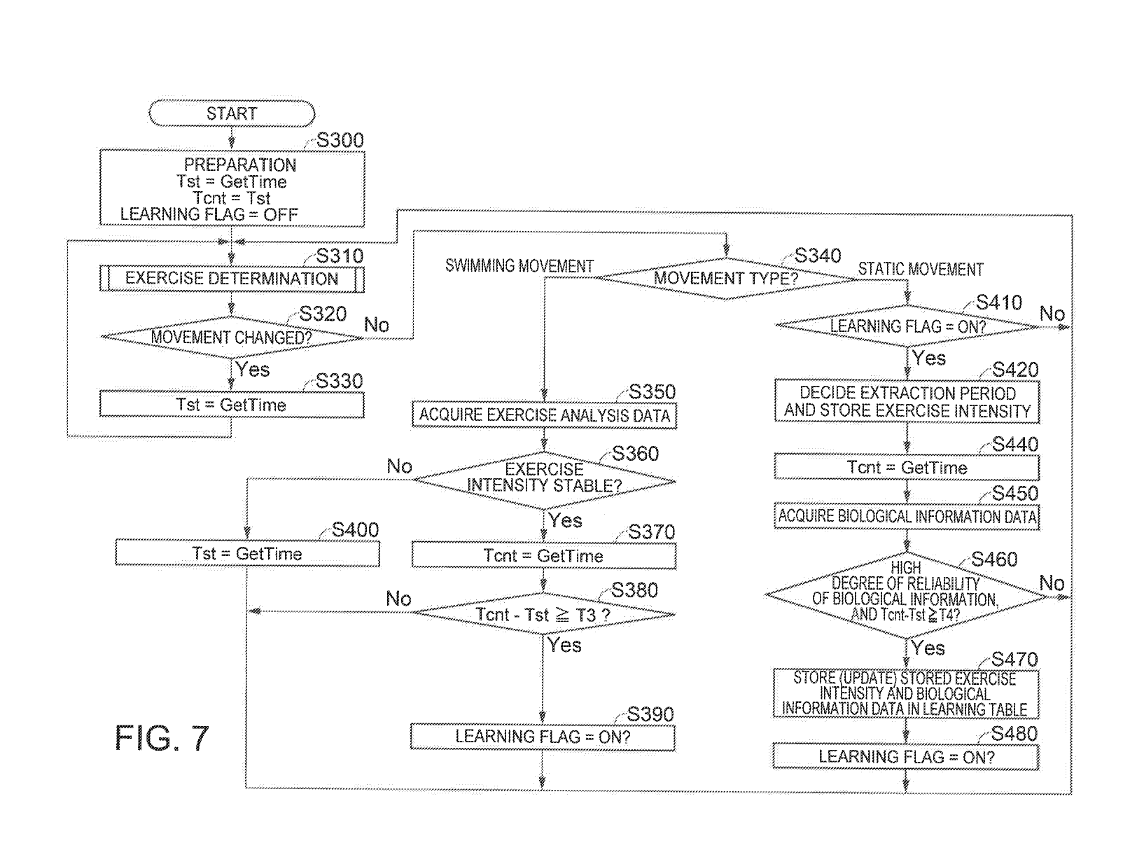

FIG. 5 is a flowchart showing the flow of the display control processing. FIG. 6 is a flowchart showing the flow of the summary information display processing. FIG. 7 is a flowchart showing the flow of the learning processing.

The flows shown in FIGS. 5, 6 and 7 are flows of processing realized by the processing unit 50 reading and executing the program 83 stored in the storage unit 70. The flow shown in FIG. 5 is equivalent to a notification control method. The flow shown in FIG. 7 is equivalent to a generation method. The flows shown in FIGS. 5, 6 and 7 are equivalent to a control method. The display processing in the flows below are equivalent to notification processing.

### Display Control Processing

In Step S10, preparation is carried out. Specifically, a variable Tst and a variable Tcnt which store time data used in the subsequent steps in this flow are initialized. “GetTime” indicates a function to acquire the current time. “Variable Tst=GetTime” expresses that the time when Step S10 is executed is stored as the variable Tst.

In Step S20, exercise determination is carried out. Specifically, a body motion signal is acquired from the body motion detection unit 10, and biological information is acquired from the biological information detection unit 16. Based on the body motion signal, the action and movement state of the user are acquired. As the movement state, a swimming movement, a turn movement, or a static movement is determined. In the case of the swimming movement, information of exercise type such as swimming style and exercise intensity such as stroke pitch is acquired as well. The explanation of exercises other than the swimming movement is omitted from this flow.

In Step S30, whether the movement is changed or not is determined. Specifically, whether the movement state determined in Step S20 is changed from the previous movement state or not is determined. More specifically, the content of the previous movement state stored in the internal variable and the content of the movement state determined in Step S20 immediately before are compared with each other. If these contents do not coincide with each other, it is determined that the movement is changed (Yes in Step S30) and the processing goes to Step S40. If these contents coincide with each other, it is determined that the movement is not changed (No in Step S30) and the processing goes to Step S100.

In Step S40, the movement type after the change is determined. Specifically, if the movement type after the change (exercise type in the movement state) is a swimming movement, the processing goes to Step S50. If the movement type after the change is a turn movement or a static movement, the processing goes to Step S60.

In Step S50, the display unit 20 is not driven (display-off state). The processing in Step S50 is equivalent to the processing of switching to the display-off 20b by the control C1 shown in FIG. 2.

From Step S60 to Step S80, the display unit 20 is driven (Step S60), exercise analysis data and biological information data are displayed on the display unit 20 (Step S70), and the current time is stored as the variable Tst (Step S80). In the variable Tst, the time when display information is displayed on the display unit 20 is stored. The processing in Steps S60 to S80 is equivalent to the processing of switching to the display-on 20c and thus displaying display information by the controls C2 and C3 shown in FIG. 2.

Steps S100 to S130 are the processing in the case where a turn movement or a static movement is continued. If the processing goes to Step S100 based on the determination in Step S30 that the movement state is not changed, and this unchanged movement state is determined as a turn movement or a static movement (Yes in Step S100), the processing goes to Step S110. If the movement state is neither a turn movement nor a static movement (No in Step S100), that is, the movement state is a swimming movement, the processing shifts to Step S20.

In Step S110, the current time is acquired and stored as the variable Tcnt.

In Step S120, whether the time obtained by subtracting the variable Tst from the variable Tcnt is equal to or longer than the period T1, or not, is determined. Specifically, since the variable Tst is the time when display information is displayed on the display unit 20, the time obtained by subtracting the variable Tst from the variable Tcnt is the time elapsed during which display information is displayed on the display unit 20. If the time elapsed is equal to or longer than the period T1 (Yes in Step S120), the processing goes to Step S130 and the display unit 20 is switched to the non-driven state. If the time elapsed is shorter than the period T1 (No in Step S120), the processing shifts to Step S20 in the state where the information display on the display unit 20 is continued. The processing in Steps S100 to S130 is equivalent to the processing of switching the display unit 20 from the display-on 20c to the display-off 20b by the control C4 after the lapse of the period T1 shown in FIG. 2.

In this way, the timing when the movement state is changed to a turn movement or a static movement is determined (Steps S20 to S40), and if the movement state is changed to a turn movement or a static movement, display information is displayed on the display unit 20 (Steps S60 to S80). The timing when the movement state is changed to a swimming movement is determined (Steps S20 to S40) and the display unit 20 is switched to the display-off state (Step S50). If display information is displayed on the display unit 20 and the turn movement or the static movement is continued, the display unit 20 is switched to the display-off state after the lapse of the period T1 (Steps S100 to S130).

### Summary Information Display Processing

In Step S200, preparation is carried out. Specifically, a variable Tst and a variable Tcnt which store time data used in the subsequent steps in this flow are initialized. In this step, the current time is stored in order to initialize the variable Tst and the variable Tcnt storing time.

In Step S210, whether an exit from the water is detected or not is determined. Specifically, pressure data is acquired from the pressure sensor 13 of the body motion detection unit 10, and whether a changed state from a water pressure value to an air pressure value is continued for a predetermined time or not is determined. If the changed state is continued for the predetermined time, it is determined that an exit from the water is detected (Yes in Step S210) and the processing goes to Step S220. Unless an exit from the water is detected (No in Step S210), this step is repeated.

In Step S220, the current time is acquired and stored as the variable Tst.

In Step S230, the current time is acquired and stored as the variable Tcnt.

In Step S240, whether the time obtained by subtracting the variable Tst from the variable Tcnt is equal to or longer than the period T2, or not, is determined. If it is equal to or longer than the period T2 (Yes in Step S240), the processing goes to Step S250. If it is shorter than the period T2 (No in Step S240), the processing returns to Step S230. That is, Steps S230 and S240 are repeated until the period T2 has passed.

In Step S250, the display unit 20 is driven. In Step S260, summary information is generated and displayed on the display unit 20.

After the lapse of the period T2 following the detection of an exit from the water shown in FIG. 2 (determination 51b), the display unit 20 is switched to the display-on 20d and the screen of the summary information is displayed.

### Learning Processing

In Step S300, preparation is carried out. Specifically, a variable Tst and a variable Tcnt which store time data used in the subsequent steps in this flow are initialized by setting the current time as these variables. A learning flag, which is one of the variables similarly used in the subsequent steps, is initialized to OFF. The learning flag is set to ON if learning is possible.

In Step S310, exercise determination is carried out. In this step, processing similar to Step S20 is carried out and the movement state is determined as a swimming movement, a turn movement or a static movement. In the case of the swimming movement, information of exercise type such as swimming style and the exercise intensity such as stroke pitch is acquired.

In Step S320, whether the movement is changed or not is determined. Specifically, whether the movement state determined in Step S310 is changed from the previous movement state or not is determined. If the movement state is changed (Yes in Step S320), the processing goes to Step S330. If the movement state is not changed (No in Step S320), the processing goes to Step S340.

In Step S330, the current time is stored as the variable Tst. The variable Tst stores the time of the timing when the movement state is changed.

Steps S340 to S480 are steps repeated in the case where the movement is not changed, that is, in the case where the same movement state is continued.

In Step S340, the movement type is determined.

Specifically, the movement type of the continued movement state is determined. If the movement type is the swimming movement, the processing in Steps S350 to S400 is carried out. If the movement type is the static movement, the processing in Steps S410 to S480 is carried out. The case where the movement type is the turn movement is omitted for the sake of convenience of the description.

Steps S350 to S400 are the processing of determining whether learning on the exercise intensity (for example, stroke pitch) of the exercise type (for example, the front crawl) is possible during the swimming movement or not.

In Step S350, exercise analysis data is acquired. Since the exercise analysis data is stored as the exercise analysis data 75 in the storage unit 70 during the swimming movement, data of the exercise intensity stored in time series is acquired from there. The data of the exercise intensity is, for example, data of the stroke pitch calculated every second.

In Step S360, whether the exercise intensity is stable or not is determined. Specifically, a moving average value of the data of the exercise intensity approximately for the most recent five seconds is calculated and the moving average is compared with the latest exercise intensity. If the difference is below a predetermined threshold, the exercise intensity is determined as stable. If the difference is equal to or above the predetermined threshold, the exercise intensity is determined as not stable (unstable). If the exercise intensity is determined as stable (Yes in Step S360), the processing goes to Step S370. If the exercise intensity is determined as unstable (No in Step S360), the processing goes to Step S400. In Step S400, the current time is stored as the variable Tst and the processing shifts to Step S310.

In Step S370, the current time is stored as the variable Tcnt.

In Step S380, whether the period obtained by subtracting the variable Tst from the variable Tcnt is equal to or longer than the period T3, or not, is determined. If the period is equal to or longer than the period T3 (Yes in Step S380), the processing goes to Step S390. If the period is shorter than the period T3 (No in Step S380), the processing shifts to Step S310.

In Step S390, the learning flag is set to ON. This indicates that learning is possible on the exercise type and exercise intensity of the exercise continued from the time of the variable Tst.

The period T3 used in the determination in Step S380 is a period for determining whether the exercise intensity of the exercise of the exercise type carried out in the movement state M4 shown in the example of FIG. 3 is stable or not. The processing in Steps S360 to S380 is carried out. If the result of the determination in Step S380 is Yes, it means that the state where the exercise intensity in the swimming movement is stable is continued for the period T3 or longer.

In this way, in the processing in Steps S350 to S400, it is determined that learning can be done (learning flag is switched ON) if the exercise is carried out with stable exercise intensity during the swimming movement.

Steps S410 to S480 are the processing of learning, during the static movement, biological information corresponding to the exercise type and exercise intensity on which learning is determined as possible in Steps S350 to S400.

In Step S410, whether the learning flag is ON or not is determined. If the learning flag is ON (Yes in Step S410), it means that there is an exercise type and exercise intensity on which learning is possible. The processing then goes to Step S420. If the learning flag is not ON (No in Step S410), there is no exercise intensity on which learning is possible. Therefore, the processing shifts to Step S310 and the next exercise determination is carried out.

In Step S420, an extraction period during which the exercise content is stable is decided and the exercise intensity in that period is stored. Specifically, data of the exercise type and exercise intensity stored in the exercise analysis data 75 is acquired, and a period during which the exercise intensity is stable, looking back from the time when the exercise is finished, is decided as the extraction period. The determination method in Step S360 is used in the determination on whether the exercise intensity is stable or not. The length of the extraction period is at least equal to or longer than the period T3 because the period during which the exercise intensity is stable is equal to or longer than the period T3 in Step S380.

The exercise intensity to be stored may be, for example, the moving average value calculated in Step S360 or may be the minimum and maximum values of the exercise intensity during the period T3. The information of the exercise intensity like this is stored in the storage unit 70 as the exercise intensity in the extraction period. At this point, the exercise type is stored as well. The stored information of the exercise intensity and exercise type is used in Step S470, described below.

In Step S440, the current time is stored as the variable Tcnt.

In Step S450, the biological information data 73 is acquired. Here, the biological information detected during the static movement is acquired.

Also, in Step S450, the biological information data 73 in the extraction period may be acquired and the degree of reliability of the biological information may be determined. If it is determined that the degree of reliability of the biological information in the extraction period is high, the processing goes to Step S470 (skipping Step S460), using this biological information. If it is determined that the degree of reliability of the biological information in the extraction period is not high, the processing goes to Step S460.

In Step S460, whether the degree of reliability of the biological information is high and the period obtained by subtracting the variable Tst from the variable Tcnt is equal to or longer than the period T4, or not, is determined. Specifically, if the SN ratio of the biological information data acquired in Step S450 is equal to or above a predetermined threshold, the degree of reliability is determined as high. The variable Tst is the time stored in Step S330. As the variable Tst, the time when the movement is switched to the static movement is stored. Therefore, the period obtained by subtracting the variable Tst from the variable Tcnt is the period during which the static movement is continued.

In Step S460, biological information is detected in the static movement state. If the degree of reliability of the detected biological information is high and the period T4 or longer has passed from the start of the static movement (Yes in Step S460), the processing goes to Step S470. Otherwise (No in Step S460), the processing shifts to Step S310.

In Step S470, the stored exercise intensity and biological information data are stored (updated) in the learning table 80. Specifically, the exercise intensity of the exercise type in the extraction period stored in Step S420 is stored in the learning table 80, in association with the biological information data acquired in Step S450. In the learning table 80, the exercise intensity of the exercise type acquired during the swimming movement in the extraction period and the biological information data acquired during the static movement are stored in association with each other. If the degree of reliability of the biological information in the extraction period is high in Step S460, the exercise intensity of the exercise type in the extraction period and the biological information data in the extraction period are stored in the learning table 80 in association with each other.

In Step S480, the learning flag is set to OFF. Since the update of the learning table 80 is finished, the learning flag is set to OFF in preparation for the learning corresponding to the exercise type and exercise intensity of the swimming movement to be carried out next.

In this way, in the processing in Steps S410 to S480, if the degree of reliability of the biological information in the extraction period is not high, the biological information data after the switching to the static movement is used and stored in the learning table 80.

As described above, the wearing device 1 according to the embodiment can achieve the following effects.

The wearing device 1 can detect the body motion signal of the user and can detect the action and movement state of the user, by the body motion detection unit 10, the body motion signal acquisition unit 51, and the exercise analysis unit 55. The wearing device 1 also detects the biological information of the user by the biological information detection unit 16 and the biological information acquisition unit 53. The display control unit 57 of the wearing device 1 generates display information which the user is notified of, based on the information acquired from the exercise analysis unit 55 and the biological information acquisition unit 53.

The exercise analysis unit 55 detects a movement state such as a turn movement, a static movement, or a swimming movement. At the time of the swimming movement, the exercise analysis unit 55 determines the exercise type and calculates the exercise intensity.

When the turn movement or the static movement is detected by the exercise analysis unit 55, the processing by the display control unit 57 is started and the generated display information is displayed on the display unit 20. When the swimming movement is detected by the exercise analysis unit 55, the processing by the display control unit 57 is started and the display unit 20 is switched to the non-driven state and thus the display-off state.

The wearing device 1 can detect the movement state of the user and can control the display-on and display-off of the display unit 20 according to the content of the detected movement state. Therefore, since the wearing device 1 can perform such control, the electricity consumption for driving the display unit 20 can be restrained, compared with the case where display information is constantly displayed on the display unit 20. Also, in the wearing device 1, the display unit 20 is switched to the display-off state when the swimming movement is detected. It is difficult for the user to swim while visually recognizing the displaying unit 20 during the swimming movement. There is very little effect even if the display on the display unit 20 is off during this time. In this way, in the wearing device 1, electricity consumption can be restrained without affecting the user-friendliness. Therefore, in the wearing device 1, the electricity consumed can be efficiently controlled by performing drive control of the display unit 20 in response to the action of the user.

Moreover, the learning unit 59 of the wearing device 1 stores the exercise intensity during the swimming movement and the biological information measured after the end of the swimming movement, in association with each other in the learning table 80. Referring to the learning table 80, the biological information corresponding to the exercise intensity during the swimming movement can be estimated. In the wearing device 1, if the biological information during the swimming movement cannot be acquired (detected), the biological information can be estimated referring to the learning table 80, and display information can be generated using the estimated biological information. Therefore, even if the biological information during the swimming movement cannot be measured stably, the biological information corresponding to the exercise state carried out by the user can be displayed.

The invention is not limited to the embodiment described above. Various changes and improvements can be added to the embodiment. Modifications will be described below.

### Modification 1

In the foregoing embodiment, the body motion detection unit 10 includes the acceleration sensor 11, the pressure sensor 13, and the gyro sensor 15. However, the body motion detection unit 10 need not necessarily include all these sensors. For example, the body motion detection unit 10 may include at least one of the acceleration sensor 11 and the gyro sensor 15 in order to analyze the movement of the user by the processing unit 50. Also, two external terminals exposed from the strap 5 of the wearing device 1 may be provided, and an entry into the water and an exit from the water may be detected, based on the presence or absence of electrical continuity between the two external terminals. In that case, the pressure sensor 13 need not necessarily be provided.

Modification 2

In the foregoing embodiment and modification, the biological information detection unit 16 has the pulse sensor 17 in order to detect pulse rate as the biological information. However, as the biological information, SpO2 (blood oxygen concentration), calories burned, body temperature and the like may be detected in addition to pulse rate. Specifically, if a sensor (light receiving element) which casts red light or infrared light and receives its reflected light is provided, SpO2 can be detected. Also, if a temperature sensor which detects body temperature in contact with the skin surface of the wrist WR or a contactless temperature sensor which detects infrared rays radiated from the wrist WR or the user's skin and thus detects body temperature is provided, body temperature can be detected. As for calories burned, the exercise intensity may be calculated using the correlation between changes in pulse rate detected by the pulse sensor 17 and changes in oxygen intake, and the calories burned corresponding to the exercise intensity can be referred to.

### Modification 3

In the foregoing embodiment and modifications, the display control unit 57 uses the average pulse rate or the maximum pulse rate, as the information calculated using the biological information data 73. However, the information is not limited to the average pulse rate or the maximum pulse rate. Any information calculated using the biological information data 73 may be employed.

For example, an exercise time in a fat burning zone, an exercise time in a muscle building zone or the like may be employed. The fat burning zone is a pulse rate range effective for fat burning. The muscle building zone is a pulse rate range effective for muscle building. These ranges are calculated using pulse rate data stored in time series in the biological information data 73.

For example, information of an indicator indicating the degree of relaxation and tension may be employed as well. The indicator indicating the degree of relaxation and tension is derived based on the correlation between the activity status of sympathetic nerves and parasympathetic serves of the heart and the status of change in pulse rate.

For example, information about degree-of-fatigue information may be calculated as well. The degree-of-fatigue information is an indicator indicating the degree of recovery from the physical load accumulated during training. The degree of recovery from fatigue can be estimated by measuring the activity state of autonomic nerves, based on the correlation between the degree of fatigue, the active state of autonomic nerves, and the lowest pulse rate (pulse rate at rest, and pulse rate during sleep or at the time of waking up). Here, the activity state of autonomic nerves is calculated, based on the distribution state of HRV (heart rate variability) indicating fluctuations in pulse interval obtained by processing a pulse wave signal detected by the pulse sensor 17.

For example, information about the evaluation of physical strength may be calculated as well. In the evaluation of physical strength, a maximum oxygen intake calculated (estimated) based on the correlation between pulse rate and exercise intensity, or a swimming movement time to reach the maximum pulse rate may be used as an indicator of the evaluation of physical strength. Also, an indicator of endurance estimated based on pulse rate can be used as an indicator of the evaluation of physical strength.

For example, information about training effect may be calculated as well. The training effect is an indicator derived using EPOC (excess post-exercise oxygen consumption). The training effect can be estimated by calculating an EPOC value based on pulse rate and exercise intensity and comparing the EPOC value calculated after an exercise with the EPOC value in the exercise in the past. Also, the training effect may be estimated based on the magnitude of the EPOC value even in a single session of exercise.

The display control unit 57 generates a display screen and a summary information display screen, using the foregoing information, and outputs these display screens to the display unit 20.

### Modification 4

This modification will be described referring to FIG. 7.

In the foregoing embodiment and modifications, in the processing flow of the learning processing carried out by realizing the processing unit 50, if the result of the determination in Step S380 is No, the processing shifts to Step S310. However, the time until the period T3 passes may be displayed on the display unit 20 before the processing shifts to Step S310.

Specifically, a period TX may be calculated by the following equation (1), and display information of a text such as “Continue swimming for another TX seconds” may be generated and outputted to the display unit 20.

Period TX=period T3−(variable Tcnt−variable Tst) (1)

The period TX is equivalent to the remaining time before reaching the third period.

Also, instead of generating the display information of the text as described above, control information such that the display unit 20 is fully switched on during the time until the period T3 passes, whereas the display unit 20 is switched off after the lapse of the period T3, may be used as display information. In the display unit 20, the liquid crystal display device is fully switched on and switched off according to the content of the control information like this. Thus, during the time when the display unit 20 is fully switched on, the user, while swimming, can recognize that the swimming movement should be continued.

According to this modification, since the user is made to continue the swimming movement while being aware of the display information, the exercise types stored in the learning table 80 can be increased. The learning table 80 can support various different exercise types.

### Modification 5

This modification will be described referring to FIG. 7.

In the foregoing embodiment and modifications, in the processing flow of the learning processing carried out by realizing the processing unit 50, if the result of the determination in Step S460 is No, the processing shifts to Step S310. However, the time until the period T4 passes may be displayed on the display unit 20 before the processing shifts to Step S310.

Specifically, a period TY may be calculated by the following equation (2), and display information of a text such as “Continue the static movement for another TY seconds” may be generated and outputted to the display unit 20.

Period TY=period T4−(variable Tcnt−variable Tst) (2)

The period TY is equivalent to the time during which the first movement state is to be maintained.

With the display information like this, the user can be made to maintain the static movement and biological information with a high degree of reliability is more likely to be detected. The biological information related to the exercise types stored in the learning table 80 can be increased.

### Modification 6

In the foregoing embodiment and modifications, biological information is detected by the biological information detection unit 16 during the swimming movement. However, the biological information detection unit 16 may be set in a non-driven state during the entirety or a part of the swimming movement, and therefore there may be a period during which biological information is not detected. Specifically, if biological information cannot be detected during the swimming movement, the biological information can be estimated from the determined exercise type, based on the body motion signal and using the learning table 80. Therefore, even if the biological information detection unit 16 is set in the non-driven state in order to save the electricity to drive the biological information detection unit 16 during the swimming movement, the biological information at that time can be estimated is the body motion signal is detected.

In this way, in the wearing device 1, electricity consumption can be restrained by controlling not only the display unit 20 but also the biological information detection unit 16 into the non-driven state during the swimming movement.

### Modification 7

The exercise in the water in the foregoing embodiment and modifications may be various swimming styles such as the sidestroke, the dog paddle, or Japanese traditional swimming styles, or may be underwater swimming styles such as the dolphin kick. Even in the case of these swimming styles, the exercise analysis unit 55 can achieve effects similar to those in the embodiment by detecting the turn movement or the static movement as the first movement state.

### Modification 8

The exercise in the water in the foregoing embodiment and modifications is not necessarily limited to the exercise of going forward and backward in the swimming pool. For example, water aerobics may be employed as well. In this case, the exercise analysis unit 55 can detect the static movement as the first movement state and can achieve effects similar to those in the embodiment. In the swimming movement, the stroke pitch is employed as the exercise intensity. However, in water aerobics, for example, the exercise analysis unit 55 may calculate the amount of movement change per unit time based on the body motion signal outputted from the acceleration sensor 11 or the gyro sensor 15 and use this value as the exercise intensity. Also, the index of exercise intensity corresponding to the amount of movement change (METs) may be calculated and the exercise intensity may be indicated by this value.

### Modification 9

In the foregoing embodiment and modifications, the display unit 20 is described as an example of the notification unit. However, the notification unit is not limited to the display unit 20 and may be an audio output unit and a vibration unit or the like.

The audio output unit is an audio output device which includes a speaker, a piezoelectric vibrator or the like and which gives various notifications based on an output signal inputted from the processing unit 50. The audio output unit converts notification information to be given to the user into audio output information such as a buzz, a speech sound, or a combination of sounds with different frequencies, and outputs this audio output information. The audio output unit may include an earphone socket and a short-range wireless communication unit. In this case, an audio output signal may be transmitted to the earphone or headphone worn by the user.

The vibration unit is a piezoelectric vibrator or a micro vibration motor or the like. The vibration unit is driven based on a signal inputted from the processing unit 50 and causes the wearing device 1 to vibrate in various different vibration patterns. The processing unit 50 selects a vibration pattern corresponding to the notification information to be given to the user, and outputs a signal of the vibration pattern to the vibration unit.

The audio output unit and the vibration unit may output an audio output or vibration if the state where the exercise content is stable is continued for the period T3 or longer (if the result of the determination in Step S380 is Yes) in the processing flow of the learning processing shown in FIG. 7.

The audio output unit and the vibration unit may output an audio output or vibration if the state where the static movement is stable is continued for the period T4 or longer (if the result of the determination in Step S460 is Yes) in the processing flow of the learning processing shown in FIG. 7.

The audio output unit and the vibration unit may output an audio output or vibration at the timing when display information that should be displayed is displayed on the display unit 20.

With the audio output outputted from the audio output unit or the vibration outputted from the vibration unit in this way, the user can easily notice the notification content described above, via a stimulus to the body due to the vibration or the audio output to the auditory sense during the exercise.

### Modification 10

In the foregoing embodiment and modifications, the stroke pitch is described as an example of the exercise intensity of the exercise type. However, the exercise intensity is not limited to the stroke pitch. Any indicator correlated with the exercise intensity may be employed. For example, the frequency and power spectrum generated with the movement calculated from the analysis information of the body motion signal, the amount of movement and calories burned per unit time, or METs may be employed as well.

Information about the indicator correlated with the exercise intensity as described above may be stored in the learning table 80.

### Modification 11

In the foregoing embodiment and modifications, as a way of using the learning table 80, an example of calculating the pulse rate corresponding to the missing part of the biological information data 73 is described. However, this use is not limiting.

For example, the pulse rate can be calculated using the learning table 80 and based on the body motion signal detected successively by the body motion detection unit 10. The use like this enables presentation of the pulse rate of the user even if the wearing device 1 is not provided with the biological information detection unit 16 (pulse sensor 17).

In the case where the wearing device 1 is provided with the biological information detection unit 16 (pulse sensor 17), the pulse rate thus calculated based on the body motion signal can be used for the signal analysis processing in the pulse sensor 17. This use enables an increase in the degree of reliability of the pulse rate detected by the pulse sensor 17.

In the description of the foregoing embodiment and modifications, swimming is employed as a specific example in order to facilitate the understanding of the invention. However, this is not limiting. For example, the invention can also be applied to other sport such as sprints, gymnastics, aerobics, tennis, golf, or soccer. The wearing device 1 can achieve effects similar to those in the foregoing embodiment and modifications, by defining the movement state where the user is concentrating on the exercise, as the second movement state (movement state that is different from the first movement state) and defining the movement state where the user is more likely to notice the notification information given by the wearing device 1, as the first movement state.

What is claimed is:

1. A wearable device comprising:
a biological information detector which detects biological information of a user;
a body motion detector which detects a body motion signal related to a movement of the user;
a processor configured to generate notification information based on at least one of the biological information and the body motion signal; and
a notification device which notifies the user of the notification information,
wherein the processor performs processing in which the notification device is configured to notify the user of the notification information if the notification information is determined as a first movement state, based on the body motion signal of the user, and the processor performs processing in which the notification of the notification information by the notification device is stopped if the notification information is determined as a second movement state that is different from the first movement state.

2. The wearable device according to claim 1, wherein the processor stops the notification of the notification information if a first period has passed after the processing in which the notification device is made to notify the user of the notification information.

3. The wearable device according to claim 1, wherein the processor stops the notification of the notification information by the notification device if a transition occurs from the first movement state that is detected to the second movement state is detected.

4. The wearable device according to claim 1, wherein the first movement state includes a static movement in swimming.

5. The wearable device according to claim 4, wherein the second movement state includes at least one of swimming movement, water walking, and water aerobics.

6. The wearable device according to claim 1, wherein the biological information detected by the biological information detector includes at least a pulse rate.

7. The wearable device according to claim 4, wherein the notification information includes at least one type of information, of at least one of a maximum pulse rate and an average pulse rate over a one-way distance from a previous turn movement to a current turn movement, a number of strokes taken, a stroke pitch, a stroke length, and a swimming time.

8. The wearable device according to claim 4, wherein the notification information includes at least one type of information, of a cumulative number of turns taken and a cumulative swimming distance.

9. The wearable device according to claim 4, wherein the body motion detector includes at least one of an acceleration sensor, a pressure sensor, and a gyro sensor.

10. The wearable device according to claim 9, wherein an entry into the water and an exit from the water of the wearable device worn by the user is detected, using the pressure sensor.

11. The wearable device according to claim 10, wherein the processor performs processing in which the notification device is made to notify the user of summary information if a second period has passed after a transition of the wearable device from the entry into the water to the exit from the water.

12. The wearable device according to claim 11, wherein the summary information includes at least one of an average pulse rate, a maximum pulse rate, a physical strength evaluation, a training effect, and degree-of-fatigue information, and at least one of a swimming time, a swimming distance, a number of turns taken, a swimming style, calories burned, an average SWOLF, and a best time recorded over a one-way distance between turn movements.

13. A wearable device comprising:
a biological information detector which detects biological information of a user;
a body motion detector which detects a body motion signal related to a movement of the user;
a processor configured to generate notification information based on at least one type of the biological information and the body motion signal; and
a notification device which notifies the user of the notification information,
wherein the processor is configured to:
determine an exercise type carried out by the user, based on the body motion signal detected by the body motion detector,
acquire the biological information detected by the biological information detector in the first movement state if an exercise of the exercise type is continued for a third period or longer and a first movement state is detected based on the body motion signal,
generate relation information between the exercise type and the biological information that is acquired, and
estimate the biological information based on the relation information.

14. The wearable device according to claim 13, wherein the processor is configured to generate information indicating a remaining time before reaching the third period.

15. The wearable device according to claim 13, wherein the notification device notifies the user by at least one of vibration or an audio output when the third period is reached.

16. The wearable device according to claim 13, further comprising
a memory which stores the relation information between an exercise intensity of the exercise type and the biological information detected in the first movement state, in association with the exercise type.

17. The wearable device according to claim 13, wherein the processor is configured to determine the biological information of the user at the current time, based on the body motion signal of the user detected by the body motion detection unit, with reference to the relation information.

18. The wearable device according to claim 13, wherein the exercise type includes at least one type of front crawl, breaststroke, backstroke, butterfly stroke, and water walking.

19. A control method comprising a notification control method and a generation method,
the notification control method including:
acquiring biological information about a user;
acquiring a body motion signal relating to a movement of the user;
performing notification processing in which the user is notified of notification information;
generating the notification information, based on at least one type of information, of the biological information and the body motion signal;
detecting a movement state based on the body motion signal of the user and performing processing in which the notification of the notification information is executed by the notification processing if a first movement state included in the movement state is detected; and
performing processing in which the notification of the notification information is stopped if the movement state that is different from the first movement state is detected, and
the generation method including:
determining an exercise type carried out by the user, based on the body motion signal;
acquiring the biological information in the first movement state, if an exercise of the exercise type is continued for a third period or longer and the first movement state is detected based on the body motion signal; and
generating relation information between the biological information that is acquired and the exercise type that is determined.

20. A wearable device comprising a processor configured to execute a program recorded in a computer-readable recording medium,
wherein the processor is configured to:
acquire biological information about a user from a biological information detector,
acquire a body motion signal relating to a movement of the user from a body motion detector,
generate notification information to notify the user of, based on at least one type of information, the biological information and the body motion signal,
detect a movement state of the user based on the body motion signal, and execute notification of the notification information if a first movement state included in the movement state is detected,
stop the notification of the notification information if the movement state that is different from the first movement state is detected,
determine an exercise type carried out by the user, based on the body motion signal,
acquire the biological information in the first movement state, if an exercise of the exercise type is continued for a third period or longer and the first movement state is detected based on the body motion signal, and
generate relation information between the biological information and the exercise type that is determined.
